(19) United States
(12) Patent Application Publication
(10) Pub. No.: US 2003/0211504 A1
(43) Pub. Date: Nov. 13, 2003

(54) Methods for identifying nucleic acid polymorphisms

(76) Inventors: Kim Fechtel, Arlington, MA (US); Shashi Prabhakar, Burlington, MA (US); Hui Huang, Newton, MA (US); Michael G. FitzGerald, Waltham, MA (US); Joann Prescott-Roy, Concord, MA (US); Michelle Runge, Bedford, NH (US); Huajun Wang, Newton, MA (US); Rene Lee Gibson, Bedford, MA (US)

Correspondence Address: GENOME THERAPEUTICS CORPORATION, 100 BEAVER STREET, WALTHAM, MA, 02453, US

(21) Appl. No.: 10/268,058

(22) Filed: Oct. 9, 2002

Related U.S. Application Data
(60) Provisional application No. 60/328,513, filed on Oct. 9, 2001.

(51) Int. Cl. C12Q 1/68; G06F 19/00; G01N 33/48; G01N 33/50

(52) U.S. Cl. 435/6; 702/20

(57) ABSTRACT

The invention provides an automated method of identifying a plurality of different polymorphisms within two or more related nucleic acid sequences. The method consists of: (a) obtaining a data set comprising a nucleic acid sequence assembly and a plurality of sequence characteristic parameters associated with said assembly; (b) indexing said nucleic acid assembly and said plurality of sequence characteristic parameters in a database; (c) selecting a region of said nucleic acid assembly having sequence characteristic parameters indicative of a polymorphic sequence, and (d) displaying two or more nucleic acid sequences of said region, said two or more sequences identifying different polymorphisms within said nucleic acid assembly. Also provided is a method of identifying a nucleic acid containing an indel region within a set of related nucleic acid sequences. The method consists of comprising: (a) dentifying a nucleic acid within two or more related nucleic acid sequences suspected of containing an indel region, said nucleic acid containing one or more regions having a plurality of polymorphisms, and (b) determining the occurrence of two or more criteria indicating the presence of an indel region associated with said one or more regions having a plurality of polymorphisms, said occurrence characterizing said nucleic acid as containing an indel region. Further provides is a method of determining the sequence of an allele containing an indel region within a set of related nucleic acid sequences. The method consists of comprising: (a) identifying a nucleic acid containing an indel region within two or more related nucleic acid sequences; (b) generating a consensus sequence within said indel region for said two or more related nucleic acid sequences; (c) identifying a matching string to said consensus sequence within at least one of said two or more related nucleic acid sequences, and (d) subtracting said consensus sequence from said two or more related nucleic acid sequences, the presence or absence of a unique sequence in one of said related nucleic acid sequences indicating the presence of an actual indel region. The invention additionally provides an automated system for identifying a plurality of different polymorphisms within two or more related nucleic acid sequences. The system consists of: (a) a sample submission module capable of transmitting data; (b) a core statistics loading and post processing module containing sequence characteristic parameters; (c) an assembly module capable constructing sequence assemblies from sequence database extracted data; (d) a SNP prospector module capable of identifying polymorphisms; (e) a polymorphism loader submodule capable of parsing polymorphic region sequence and sequence characteristic parameters from sequence assemblies; (f) a SNP database structured to contain the information produced in steps (a) through (e), and (g) an output module for display or further manipulation of specified data in step (f).

### BACKGROUND OF THE INVENTION

This invention relates generally to genomics and related bioinformatic methods for processing large amounts of nucleic acid sequence information and, more specifically to methods of identifying polymorphic sites and regions within a repertoire of related nucleic acid sequences.

The human genome project has resulted in the generation of enormous amounts of DNA sequence information. The generation of this information and achievement of the complete sequencing of the human genome has required numerous technical advances both in sample preparation and sequencing methods as well as in data acquisition, processing and analysis. During the project's quick evolution, it has brought to fruition the scientific fields of genomics, proteomics and bioinformatics. As a result, a complete draft sequence of the human genome was published in February of 2001. Moreover, in developing and improving processes for sequencing, processing and analysis of genomic quantities of sequence information, the complete genome sequences of at least two different eucaryotic organisms have now been reported with numerous others approaching completion.

Automated DNA sequencing procedures have been developed that require essentially little to no human intervention outside of sample preparation. For example, computerized robotics generate and perform sequencing reactions and the resulting signals are detected by sensors which are read into a computer. Algorithms and software are available which analyze and process signal from noise in order to detect the nucleotide sequence for a corresponding reaction. The signals can then be transformed into a graphical display or other readout formats convenient for the user.

The number and rate of different reactions which can be performed currently exceeds hundreds of thousands of bases per day. Analyzing and processing such information into useful strings that reflect the nucleotide sequence of the genes and chromosomes from which they were derived can be performed by assembly or alignment algorithms and their corresponding computer executable code. Such programs compare and organize a multiplicity of like sequences into groups and merge them into a single contiguous sting of unique nucleotides representing the sequence of a DNA strand.

One problem with assembly of nucleic acid sequence information from an immense amount of similar sequences into a true representation of the real sequence is the occurrence of minor differences between otherwise identical sequences. For example, when aligned in a region containing a single nucleotide difference between two sequences, or two groups of sequences, the question arises as to whether the difference is real or is an artifact of experimental or computational error. True sequence differences will represent new genotypes such as a different allele for a particular gene. Although implementation of repetitive and complementary sequence routines can increase the quality sequence information, the confidence of automated base assignment at such positions is rarely error free.

Another drawback arising from minor differences between otherwise identical sequences occurs during sequence alignment and analysis. Insertion or deletion of as little as a single nucleotide can have dramatic effects on alignment results. As with single nucleotide substitutions, inclusion of an inserted or deleted sequence will result in the creation of a new genotype and similarly require an assessment of whether such a sequence is real or an artifact of the process. Additionally, however, inclusion of the inserted or deleted sequence also will result in misalignment of sequences following the inserted or deleted region. Because of the underlying computer algorithms and logic used in genomic analysis programs, misalignment in an assembly produces computational difficulties for identification of the unique sequence within an otherwise identical surrounding sequence. Therefore, minor changes between nucleotide sequences in an assembly can result in significantly different treatments of the information obtained by an automated computer process.

The above problems observed in genomic and other large scale sequence acquisition and analysis has been substantiated by the first reports of the completed draft sequence of the human genome. For example, comparison of complete drafts of the genome published by two independent groups has revealed a significant number of discrepancies. The accuracy of the complete sequence of the human and other genomes is important in the diagnosis and treatment of diseases because even a single nucleotide change within a gene can have dramatic effects on the occurrence or treatment of a disease. However, the ultimate accuracy of any sequence information obtained by such genomic and bioinformatic methods is only as good as its weakest analytical component and only as complete as its computational repertoire of available analytical components.

Thus, there exists a need for methods, computational modules and repertoires that can efficiently detect, analyze and process large amounts of related sequencing data to determine the true sequence of similar nucleic acids. The present invention satisfies this need and provides related advantages as well.

### SUMMARY OF THE INVENTION

The invention provides an automated method of identifying a plurality of different polymorphisms within two or more related nucleic acid sequences. The method consists of: (a) obtaining a data set comprising a nucleic acid sequence assembly and a plurality of sequence characteristic parameters associated with said assembly; (b) indexing said nucleic acid assembly and said plurality of sequence characteristic parameters in a database; (c) selecting a region of said nucleic acid assembly having sequence characteristic parameters indicative of a polymorphic sequence, and (d) displaying two or more nucleic acid sequences of said region, said two or more sequences identifying different polymorphisms within said nucleic acid assembly. Also provided is a method of identifying a nucleic acid containing an indel region within a set of related nucleic acid sequences. The method consists of comprising: (a) dentifying a nucleic acid within two or more related nucleic acid sequences suspected of containing an indel region, said nucleic acid containing one or more regions having a plurality of polymorphisms, and (b) determining the occurrence of two or more criteria indicating the presence of an indel region associated with said one or more regions having a plurality of polymorphisms, said occurrence characterizing said nucleic acid as containing an indel region. Further provides is a method of determining the sequence of an allele containing an indel region within a set of related nucleic acid sequences. The method consists of comprising: (a) identifying a nucleic acid containing an indel region within two or more related nucleic acid sequences; (b) generating a consensus sequence within said indel region for said two or more related nucleic acid sequences; (c) identifying a matching string to said consensus sequence within at least one of said two or more related nucleic acid sequences, and (d) subtracting said consensus sequence from said two or more related nucleic acid sequences, the presence or absence of a unique sequence in one of said related nucleic acid sequences indicating the presence of an actual indel region.

The invention additionally provides an automated system for identifying a plurality of different polymorphisms within two or more related nucleic acid sequences. The system consists of: (a) a sample submission module capable of transmitting data; (b) a core statistics loading and post processing module containing sequence characteristic parameters; (c) an assembly module capable constructing sequence assemblies from sequence database extracted data; (d) a SNP prospector module capable of identifying polymorphisms; (e) a polymorphism loader submodule capable of parsing polymorphic region sequence and sequence characteristic parameters from sequence assemblies; (f) a SNP database structured to contain the information produced in steps (a) through (e), and (g) an output module for display or further manipulation of specified data in step (f).

### BRIEF DESCRIPTION OF THE DRAWINGS

FIG. 1 shows nucleotide sequence tracings for a homozygous allele or for heterozygous alleles containing either a single nucleotide insertion or a single nucleotide polymorphism (SNP). Also shown are nucleotide calls for a sequence containing an indel.

FIG. 2 shows genotype calls obtained from the Phrap/Polyphred automated process using high quality (FIG. 2A) and low quality (FIG. 2B) thresholds to identify polymorphic sites.

FIG. 3 shows a schematic representation for identifying nucleic acids containing indel regions and the indel region sequence.

FIG. 4 shows a diagram of an automated system for polymorphism discovery.

### DETAILED DESCRIPTION OF THE INVENTION

This invention is directed to an automated system for combining and analyzing nucleic acid sequence information to select polymorphic regions between related nucleic acid sequences. The polymorphic regions include single nucleotide polymorphisms (SNPs), insertions and deletions as well as substitutions of multiple bases. The method is advantageous because nucleic acid sequence information can be indexed in a database together with parameters characterizing various attributes of the sequence information. The parameters can be used to identify, search, sort and display relevant regions of their associated nucleic acid sequence that satisfy criteria for a specific type of polymorphic sequence. The results can be manipulated or combined with other analysis, displayed for user visualization or outputted into a variety of useful formats.

The invention also is directed to a method of identifying a nucleic acid containing an insertion or deletion (indel) region among a plurality of related nucleic acids. The method of identifying a related nucleic sequence containing an indel region will also provide the nucleotide sequence of the related nucleic acid as well as the nucleotide sequence of the indel region. The method for identifying indel regions can advantageously be applied to large numbers of nucleic acid sequences in an automated process. Another advantage of the method is that it can be used in the absence of neighboring sequence information and without requiring prior predictions of the indel sequence characteristics such as its length. Therefore, the methods of the invention can be integrated into a wide variety of genomic and bioinformatics applications to obtain new and accurate nucleic acid sequence information as well as to refine, optimize and corroborate existing sequence data.

In one embodiment, the invention is directed to an automated method of identifying a plurality of different polymorphisms within related nucleic acid sequences. The polymorphisms can be single nucleotide polymorphisms. The method combines data sets for sequence characteristic parameters with that for nucleic acid sequence information in a database. The sequence characteristic parameters include information such as confidence of a polymorphic sequence, quality of the sequence, signal, noise and signal to noise ratio and are used to select the polymorphic regions of the associated nucleic acid sequences. The results of the selection and the different allelic sequences corresponding to the polymorphic regions can be displayed or further manipulated. Moreover, the methods for identifying indel regions and indel sequences also can be combined with the sequence characteristic parameters and the sequence information for additionally identifying these classes of sequences within a polymorphic region. Indel sequences can be identified independently or simultaneously with the identification of SNPs.

In another embodiment, the invention is directed to a method of determining the nucleotide sequence of an indel region within a plurality of alleles. Briefly, possible inserted and deleted sequences between two alleles are identified by tagging regions within one allele that contains polymorphic nucleotide positions or aberrant sequence alignment or quality characteristics compared to the other allele or compared to other read sequences corresponding to the alleles. Comparison of the nucleotide sequences for a plurality of read sequences corresponding to the alleles within the potential indel region will allow identification of a consensus sequence for the plurality of alleles. Determining whether the possible indel region is an actual insertion or deletion is performed by searching for a matching nucleotide strings to the consensus sequence within the possible indel region. Identification of a matching sequence allows deduction of the individual allele sequences and determination of the actual indel region sequence by subtraction of the consensus sequence from the two alleles.

In a further embodiment, the invention is directed to a computer algorithm that specifies execution of steps for identifying alleles containing an indel region and for identifying indel region sequences within a plurality of related allele sequences. The automated process of the algorithm employs the output of Polyphred for identifying regions within alleles that indicate polymorphic sites, insertions or deletions. Briefly, the automated process identifies indel regions within an allele obtained from a plurality of alleles based on local concentration of polymorphic sites, requirement for additional sequence information proximally located on both strands of a sequenced allele and the requirement for additional sequence information aberrantly located relative to other read sequences for the same region of an allele. Possible indel regions between two alleles are marked using these criteria and compared to a consensus sequence obtained from a plurality of read sequences related to the allele. Identified consensus sequences can be subtracted from the two allele sequences to distinguish the actual sequences of each allele. The unique nucleotide sequences between the two alleles constitute the actual indel region sequence.

As used herein, the term “data set” is intended to mean a combination of two or more data elements that characterize an attribute of a nucleic acid sequence. A data set can contain two or more similar or different types of data elements. For example, a data set of the invention can include two or more nucleic acid sequences. Data sets of the invention also can include, for example, a nucleic acid sequence as one data element and one or more physical, chemical or computational characteristics associated with the sequence as other data elements. A data element refers to a value or other analytical representation of factual information that describes a characteristic or property of a nucleic acid sequence or a component thereof. A data element can be represented by, for example, a number, a symbol, a hue or color, a geometric shape, a set of coordinates, a word, an alphanumeric string or any other descriptive form or form suitable for computation, analysis, or processing by, for example, a computer or other machine or system capable of data integration and analysis. A specific type of data element is a sequence characteristic parameter.

As used herein, the terms “sequence characteristic parameter” or “parameters” are intended to mean a property of a nucleic acid sequence or of a read sequence. A property of a sequence or read can include, for example, physical, chemical, statistical or computational properties as well as other associated attributes. Physical and chemical properties include, for example, a primary structure or base composition of a sequence or read. Statistical and computational properties include, for example, fluorescent trace statistics, error values, quality values, signal, noise and probabilities. Various other characteristics and attributes well known to those skilled in the art also are included within the meaning of the term so long as they provide a description or information associated with a nucleic acid sequence or with a read sequence. The description or information can be, for example, qualitatively or quantitatively describe an property or attribute of the sequence, read or component thereof. Sequence characteristic parameters can be transformed into a variety of output formats including an annotated tag for visual display.

As used herein, the term “assembly” is intended to mean the collection and fitting together of portions of a nucleic acid sequence into a contiguous sequence representation. A contiguous collection of sequence portions will be represented without redundancy and derived from non-coextensive, overlapping portions of sequence. Therefore, the term is intended to mean a linear, non-redundant electronic representation of a sequence constructed from smaller overlapping sequences or reads corresponding to the parent nucleic acid molecule.

As used herein, the term “indel” is intended to mean the presence of an insertion or a deletion of one or more nucleotides within a nucleic acid sequence compared to a related nucleic acid sequence. An insertion or a deletion therefore includes the presence or absence of a unique nucleotide or nucleotides in one nucleic acid sequence compared to an otherwise identical nucleic acid sequence at adjacent nucleotide positions. The term “indel region” as it is used herein, is intended to mean the site or location of an inserted or deleted nucleotide sequence or sequences. Insertions and deltions can include, for example, a single nucleotide, a few nucleotides or many nucleotides, including 5, 10, 20, 50, 100 or more nucleotides at any particular position compared to the related reference sequence. It is understood that the term also includes more than one insertion or deletion within a nucleic acid sequence compared to a related sequence.

As used herein, the term “single nucleotide polymorphism” or “SNP” is intended to mean a difference in nucleotide sequence between two related nucleic acids of one nucleotide at a specified position. The term therefore refers to a nucleotide substitution at a particular position compared to an otherwise identical nucleic acid sequence at adjacent nucleotide positions.

As used herein, the terms “polymorphism,” “polymorphic,” “polymorphic site” or “polymorphic region” are intended to mean a nucleotide position or positions in one nucleic acid sequence that differs compared to a related sequence. Therefore, the term refers to a relative difference in primary structure between two compared nucleic acids that are substantially related. Polymorphisms and polymorphic sites include, for example, nucleotide insertions, deletions and substitutions in one nucleic acid sequence compared to a related sequence. A polymorphic site or region includes, for example, indels and SNPs.

As used herein, the term “related” when used in reference to a nucleic acid sequence is intended to mean a nucleotide sequence that is substantially the same as a reference sequence. Related nucleic acid sequences can include, for example, gene alleles or two otherwise similar nucleic acids where one nucleic acid within a pair contains an insertion, deletion or SNP. Therefore, related nucleic acid sequences can be derived, for example, through natural processes, such as by evolution or mutation. Similarly, related nucleic acid sequences can be derived, for example, by recombinant or chemical synthesis methods, such as by engineering specific alterations within a selected nucleic acid sequence. Alternatively, related nucleic acid sequences can be derived, for example, by experimental artifacts obtained during sample preparation, processing, manipulation, sequencing or data interpretation.

As used herein, the term “consensus” is intended to mean the reduction of a nucleotide position in a multiple alignment to a single base character representing the most frequent nucleotide occurring at the referenced position. An alignment refers to a display of two or more sequences sharing matches, mismatches or gaps at each position.

As used herein, the term “unique” is intended to mean that the nucleotide sequence is unmatched in comparison to a related reference sequence in a sequence alignment. Therefore, the term includes a nucleotide sequence, or portion of a sequence, that does not have an equal comparison in the reference sequence. Unique sequences can include, for example, insertions, deletions and SNPs.

As used herein, the term “actual” when used in reference to a nucleotide sequence is intended to mean that the sequence is the authentic or genuine nucleotide sequence. The term therefore refers to the true genotype of a referenced nucleotide sequence. An actual nucleotide sequence is devoid of experimental artifacts incurred in generating its sequence.

As used herein, the term “read” or “read sequence” is intended to mean the nucleotide or base sequence information of a nucleic acid that has been generated by any sequencing method. A read therefore corresponds to the sequence information obtained from one strand of a nucleic acid fragment. For example, a DNA fragment where sequence has been generated from one strand in a single reaction will result in a single read. However, multiple reads for the same DNA strand can be generated where multiple copies of that DNA fragment exist in a sequencing project or where the strand has been sequenced multiple times. A read therefore corresponds to the purine or pyrimidine base calls or sequence determinations of a particular sequencing reaction.

As used herein, the term “automated” or “automated process” is intended to mean a self-controlled operation of an apparatus, process or system by mechanical or electrical devices, or both, that can substitute for human intervention, including cognitive decision processes. Minor human interventions which do not substantially affect the primary functions of the process are included within the definition of the term. Such minor interventions can include, for example, input and export of data, including beginning and ending data. Generally, a process is automated through the control of a computer, which is a programmable electronic device that can store, retrieve and process data. An algorithm refers a series of procedural instructions that define the automated steps of a method. In a computerized process, the algorithm defines a list of coded instructions implemented by the computer.

In large scale nucleic acid sequencing projects, immense amounts of sequence information can be generated in very short periods of time. Computer automated processes have been employed to generate and process such quantities of information within usable time frames. The accurate analysis of the information becomes important because single nucleotide differences within sequence alignments can result in assignment of a new genotype for a particular gene. Physiologically, single nucleotide differences can have dramatic effects on the occurrence and treatment of a disease. For example, one genotype or allele can result in normal phenotypes while another allele can be associated with pathogenesis. Specific examples include the different alleles for BRAC 1, BRAC 2 and for Sickle cell anemia. Identification of different alleles within individuals or within populations also is useful in, for example, the field of pharmacogenomics. Therefore, the beneficial affect of human genome sequence information to the health care industry will correlate with the attainment of accurate and reliable information at each and every position. The importance of accurate sequence information is compounded as more allelic polymorphisms are identified and their complex associations are determined. The methods of the invention are useful in efficiently identifying minor differences between sequences that are otherwise substantially identical. Such methods are useful in simple and complex systems which generate, process and analyze both small numbers of sequences as well as large numbers, including hundreds of thousands of sequences.

The invention provides an automated method of identifying a plurality of different polymorphisms within two or more related nucleic acid sequences. The method consists of: (a) obtaining a data set comprising a nucleic acid sequence assembly and a plurality of sequence characteristic parameters associated with said assembly; (b) indexing said nucleic acid assembly and said plurality of sequence characteristic parameters in a database; (c) selecting a region of said nucleic acid assembly having sequence characteristic parameters indicative of a polymorphic sequence, and (d) displaying two or more nucleic acid sequences of said region, said two or more sequences identifying different polymorphisms within said nucleic acid assembly.

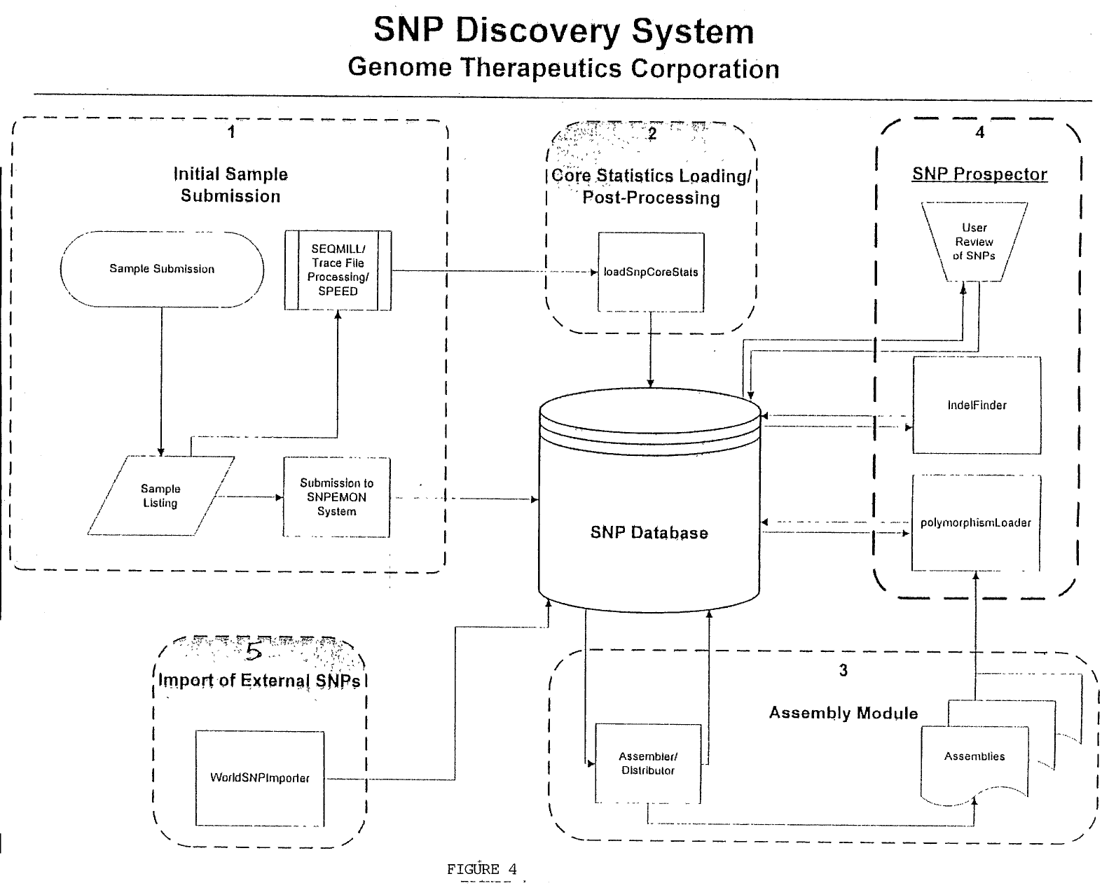

The methods of the invention for identifying a plurality of different polymorphisms can be employed independently for analysis of nucleic acid sequences. Alternatively, the methods for identifying a plurality of different polymorphisms can be used in combination with a larger sequence discovery, analysis and data management system. Such a larger discovery system is shown in FIG. 4 and described further below. The method for identifying a plurality of different polymorphisms is shown as module 4, termed “SNP Prospector,” in FIG. 4.

The methods of the invention are applicable in distinguishing true or actual genotypes from sequence errors resulting from sequencing errors, base calling errors, trace crossovers and read misalignments, for example. Additionally, the methods of the invention are applicable for identifying and determining the true genotype of various polymorphic alleles within a heterogenous set of related alleles. The methods are applicable to manual manipulations of sequence data but, more advantageously, can be employed in computerized systems for automated analysis and determination of sequence differences. Allelic differences resulting from SNPs and nucleotide insertion or deletion (indel) can be identified and distinguished between themselves and from false positives. Therefore, the methods of the invention are useful in identifying individual alleles containing a SNP or an indel region from read sequence data obtained from directly sequenced PCR (polymerase chain reaction) templates.

The methods of the invention also are applicable for identifying differences between nucleic acid sequences. Nucleotide differences between sequences can include a wide range of sizes. For example, both single and multiple nucleotide changes can be readily determined between compared nucleic acid sequences. Such changes can include, for example, nucleotide substitutions such as those found in SNPs as well as nucleotide additions and deletions such as those found in sequences containing indel regions. The procedural logic used in the methods of the invention result in accurate and reliable identification of nucleotide differences between compared sequences. Therefore, the methods are applicable to large scale sequence analysis of related nucleic acid sequences having a single or small numbers of nucleotide changes between compared sequences.

Related nucleic acid sequences of invention include, for example, nucleotide sequences that are substantially the same in at least one region of comparison. The region of comparison includes, for example, sufficient nucleotide sequence information to identify the related nucleic acid sequences and being substantially the same or even identical. Related nucleic acid sequences can be obtained from the same nucleic acid fragment or from different nucleic acid fragments. For example, multiple sequencing reactions of a common template will result in reads that contain related nucleic acid sequences. Similarly, sequencing from duplicated templates also will result in reads containing related nucleic acid sequences. Related nucleic acid sequences will similarly be obtained from substantially similar but different nucleic acids such as that derived from different alleles of the same gene or gene fragment. Multiple sequencing reactions from such allelic variants also will result in reads that contain related nucleic acid sequences. Therefore, related nucleic acid sequences of the invention can include genotypically related sequences such as occurs between allelic variants and polymorphisms as well as methodologically related sequences, such as occurs between reads from different sequencing reactions for a particular nucleic acid fragment.

Sets of related nucleic acid sequences can include a wide range of different sizes. For example, sets can include a group as small as two related sequences as well as one or more groups of hundreds or thousands or more related nucleic acid sequences. Therefore, sets can include a plurality of 2, 3, 4, 5, 6, 8, 10, 12, 15, 20 or more nucleic acid sequences as well as larger sets consisting of tens of independently determined but related sequences. For example, a set of related nucleic acids also can contain 25, 30, 40, 50 or 100 or more different read sequences as well as 200, 300, 500 or or 2000 or more different read sequences. It will be apparent to those skilled in the art that as the number of related nucleic acid sequences within a set to be compared increases, the need for performing the methods of the invention efficiently will be satisfied through automated computer process such as those described below. However, the methods of the invention can be applied to an essentially unlimited number of related nucleic acid sequences given the teachings and guidance provided herein. Therefore, the number of sets of related nucleic acid sequences will only be limited by the available computational power.

The automated method of the invention for identifying polymorphisms within related nucleic acid sequences can be performed using sequence assembly data obtained from any of a variety of programs well known to those skilled in the art. As described further below, such programs can include, for example, Phred, Phred-qual, Phrap, PolyPhred, PhredPhrap and Consed, which consist of a suite of programs that can be used to obtain sequence trace data to output viewing of assembled sequences. In automating the sequence determination, such programs also generate relevant error statistics and quality values of the read sequence information. Such information is used in identifying the most likely read sequences to combine into an assembly and to set forth where additional sequence information is suggested to be further obtained. Additionally, PolyPhred for example, functions to identify SNPs within a nucleic acid region from a plurality of different read sequence information. The automated method of the invention enhances the efficiency of polymorphism discovery by creating a data set of such information together with the nucleic acid sequence information in a searchable and manipulable database. A variety of other information and attributes, or sequence characteristic parameters, are also included in the database to enable the rapid and reliable identification of a number of different types of polymorphisms.

Generally, the nucleic acid sequence will be obtained from an electronic assembly produced from large-scale sequencing projects of a particular genomic or other nucleic acid region. However, the method of the invention also can be used with nucleic acid sequence information obtained from essentially any available source known to those skilled in the art. In addition to nucleic acid sequence information such as a plurality of reads obtained from related nucleic acids, a data set also can include one or more sequence characteristic parameters associated with the nucleic acid sequence. For example, statistical and computational properties such as fluorescent trace statistics, error values, quality values, signal, noise, probabilities and polymorphism rank can be include in a data set. Trace statistics can include, for example, peak spacing, uncalled to called ratio and peak resolution, for example. These and other sequence characteristic parameters well known to those skilled in the art can be included in a data set for use in the automated methods of the invention.

A data set containing one or more nucleic acid sequences and a plurality of sequence characteristic parameters can be indexed or combined in a database. The combination of such information can be accomplished to allow searching and manipulation of the sequence information by one or more parameters, by sequence or region thereof, or both. For polymorphism identification, for example, the sequence information can be parsed based on parameters characteristic of one or more types of polymorphisms.

For example, and as described further below, SNPs can be identified based on characteristics of lower normalize peak height and a second underlying peak at the nucleotide position in question. These characteristic can be used to parse out portions of an assembly that contain SNPs for identification of respective genotypes. Similarly, parameters characterizing indel regions and polymorphic regions in general can similarly be used to parse the associated region of an assembly. The resultant nucleic acid sequence information can be combined, for example, in a database in a format useful for further manipulation, mining or output.

Data sets containing nucleic acid sequence information and sequence characteristic parameters can be inserted into a data base in any of a variety of formats well known to those skilled in the art. For example, one useful format for nucleic acid sequences is a “phd” file. This format is used, for example, in a variety of automated sequencing system, including Phred, Phrap, PolyPhred and Consed. Phd files can be manipulated as a text file for efficient processing and output of nucleic acid sequence information. Functional equivalents of phd files, which perform or enable substantially the same activity as a phd file, also can be a useful format for nucleic acid sequences in data sets of the invention.

One format useful for processing and manipulation of sequence characteristic parameters can be, for example, an “ace” file. As with phd files, ace files similarly are employed in a variety of automated sequencing systems, including Phred, Phrap, PolyPhred and Consed. Sequence attributes and parameters which can be contained and manipulated in ace files include all of the previously described sequence characteristic parameters as well as other parameters and relevant values well known to those skilled in the art. Ace files can be manipulated in the database or produced as a visual output in the form of an annotated tag associated with the its characterized sequence. Functional equivalents of ace files, which perform or enable substantially the same activity as an ace file, also can be useful format for sequence characteristics parameters in data sets of the invention.

Through the creation or obtainment of a data set containing nucleic acid sequence information together with a wide range of sequence characteristic parameters or attributes, a manipulable database can be generated which productively associates these pieces of information. The resultant associations can be advantageously utilized for identification, training and refinement of specific combinations of parameters which yield accurate and reliable identification of polymorphic regions, SNPs, insertions, deletions or substitutions between two or more related nucleic acid sequences. Accuracy of identification of specific polymorphisms such as SNPs and indels using the automated methods of the invention can be, for example, greater than about 80%, generally greater than about 85%, and more generally greater than about 90%, 95% or 99%. At such accuracies, the methods of the invention are useful for correctly identifying the actual genotype of alleles within a plurality of related nucleic acid sequences.

One advantage of the method is that it can be employed for accurately identifying the actual respective genotypes of heterozygous alleles in the absence of corresponding homozygous nucleic acid sequence information. The actual genotype sequences can be, for example, displayed visually, output in other formats well know to those skilled in the art or inserted back into the database for further manipulation, processing or analysis. Moreover the methods of the invention are applicable for the processing of large volumes of sequence and parameter information either in series or in parallel. Therefore, the methods of the invention can, for example, process, analyze and display regions indicative of polymorphic sequences either consecutively or simultaneously for a plurality of different nucleic acid assemblies. The number of different assemblies include, for example, 5 or more, 10 or more, 20 or more, 50 or more and up to, or greater than 100 or more different nucleic acid sequences.

The invention provides a method of identifying a nucleic acid containing an indel region within a set of related nucleic acid sequences. The method consists of (a) identifying a nucleic acid within two or more related nucleic acid sequences suspected of containing an indel region, said nucleic acid containing one or more regions having a plurality of polymorphisms, and (b) determining the occurrence of two or more criteria indicating the presence of an indel region associated with said one or more regions having a plurality of polymorphisms, said occurrence characterizing said nucleic acid as containing an indel region.

As with the above described automated method for identifying a plurality of different polymorphisms within two or more related nucleic acid sequences, the methods of identifying a nucleic acid containing an indel region can be employed independently or used in combination with a larger sequence discovery, analysis and data management system, such as the discovery system shown in FIG. 4 and described further below. The methods for identifying indel regions are shown as the submodule “Indel Finder” within Module 4 of FIG. 4.

Related nucleic acids suspected of containing an indel region can include, for example, polymorphic alleles. Differentiation of the actual sequence of such heterozygous alleles in a sample can be difficult when using, for example, shotgun sequencing procedures where both allelic forms are contained in the same reaction or group of reactions. Determining the actual nucleotide sequence within indel regions has previously required the comparison of adjacent homozygous sequence regions, an estimation of indel length or direct visual scanning of the region. The methods of the invention circumvent these drawbacks and therefore can be used to determine the actual sequence of an indel region within a set of related sequences in the absence of such information and without human intervention. The methods of the invention are therefore applicable for the determination of indel region sequences in completely heterozygous allele samples, including low-frequency heterozygous alleles. The methods for identifying a nucleic acid containing an indel region can be used alone or in combination with the previously described methods of the invention for identifying a plurality of different polymorphisms.

Identification of the suspect indel region can be routinely performed by a variety of methods well known to those skilled in the art. For example, empirical or computer alignment can reveal a concentration of nucleotide sequence discrepancies within a particularized region. One useful method well known to those skilled in the art for automated determination of nucleotide sequences and sequence differences is the Phred/Phrap/Polyphred/Consed group of algorithms and computer programs. Within this computer environment, one can automatically generate base calls from sequencing traces, assemble read sequences, identify possible polymorphisms and visually display, manipulate or modify the output. Such methods and programs are described, for example, in Ewing et al., Genome Res. 8:175-185 (1998); Ewing and Green, Genome Res. 8:186-194 (1998); Nickerson et al., Nucleic Acids Res. 25:2745-2751 (1997); Rieder et al., Nucleic Acids Res. 26:967-973 (1998); Gordon et al., Genome Res. 8:195-202 (1998); and Gordon et al., Genome Res. 11:614-625 (2001) and at the URLs:genome.washington.edu and droog.mbt.washington.edu.

Another useful method well know to those skilled in the art for automated sequence determination and identification of sequence differences can be the Gap4 group of assembly algorithms and computer programs, including Trace_Diff, as described, for example, by Bonfield et al., Nuc. Acid Res. 26:3404-3409 (1998), and at the URL:mrc-lmb.cam.ac.uk/pubseq/. Numerous other methods well known to those skilled in the art also exist and can similarly be employed in the methods of the invention for identifying nucleic acid sequences suspected of containing an indel region. Such additional methods have employed, for example, the ABI SeqEd and visual comparison and the PE/ABI Factura program (Perkin Elmer Corp., Palo Alto) for identification of sequence differences between two or more related nucleic acid sequences. Tamary et al., Am. J. Hematol. 46:127-133 (1994); Jonsson et al., Am. J. Hum. Genet. 56:597-607 (1995); and Phelps et al., Biotechniques 19:984-989 (1995).

Polyphred identifies sequence differences such as SNPs by detecting both a drop in normalized peak height at a polymorphic site when fluorescent traces from heterozygous and homozygous individuals are compared, and the occurrence of a second underlying peak at the variant position. In contrast, Gap4 and Trace_Diff identifies sequence differences by fluorescent sequence trace subtraction. In the former program, sequence differences are ranked and tagged in a visual display according to an error probability representing quality values and according to log-likelihood ratios for computing matches between two read sequences. The Polyphred rankings range from 1-6, highest to lowest quality, respectively, where the recommended default ranks are 1-3. In the latter program, wild-type and mutant fluorescence-based sequencing traces are normalized and subtracted to produce a new visually displayed trace which represents only the variant positions.

Identification of nucleotide differences between related nucleic acid sequences, such as in a sequencing project assembly group, is one method for identifying regions within related nucleic acid sequences that can contain insertions or deletions. For example, alignment within an assembly group is sufficient to demonstrate that the group of read sequences are related. Base differences within the aligned sequences indicate, for example, that the divergent sequence can be a different genotype. However, because of the complexity of flourescent traces and base calls by current automated systems, it remains difficult to accurately determine the actual nucleotide sequence within the divergent area of the read sequences. Calling is the process of making a determination as to which purine or pyridine base is the most likely to be the actual base at the referenced position.

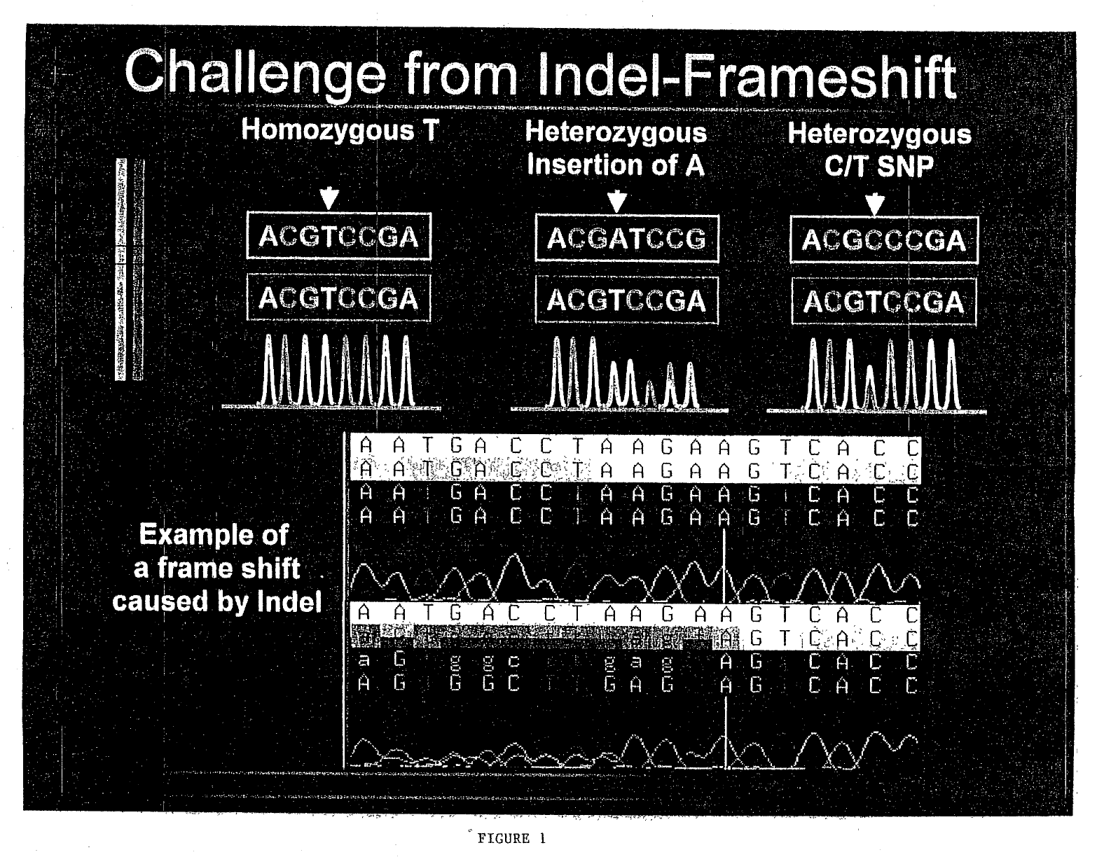

For example, FIG. 1 is a schematic diagram of flourescent traces for an eight nucleotide sequence where the read sequences are obtained from individuals that are either homozygous or heterozygous within the represented region. A color drawing identical to FIG. 1 is attached as Exhibit A. The heterozygous individuals contain a single nucleotide change within the represented eight nucleotide sequence. The middle panel, for example, represents a heterozygous insertion of a single nucleotide while the right panel represents a single nucleotide substitution or SNP. Representative flourescent traces are shown below each nucleotide sequence. As shown in the left panel, the homozygous sequence is readily discernable from the flourescent traces and can be called manually or, more usually, by automated computer systems using programs such as Phred. Similarly, the SNP variant also can be called with a reasonable degree of confidence, or alternatively, it can be readily determined that the sequence contains a variation at one position.

In contrast, comparison of the heterozygous insertion of a single nucleotide results in a complex flourescent trace. From such a trace, it can be unclear whether the analyzed sequence contains an insertion or a deletion and what is the size of the differing sequence. It can also be unclear whether the analyzed sequence contains multiple, adjacent substitutions instead of an indel sequence. For example, in the middle panel of FIG. 1, one of the heterozygous sequences contains a single nucleotide insertion at the fourth position. However, comparison of the two heterozygous traces results in all positions beginning at the insertion showing a variation. The bottom panel of FIG. 1 is a visual display of the base calls from such a heterozygous sequence analysis, which indicates the variation by the colored tags.

Nucleic acids suspected of containing an indel region can be identified by, for example, a correlation with one or more regions exhibiting a plurality of polymorphisms can be confirmed to contain an actual indel sequence by further associating the polymorphic region or regions with at least two criteria which indicate the presence of an indel region. Associating a polymorphic region with more than two criteria can increase confidence further in the initial confirmation of the actual indel sequence.

Identification of local concentration of sequence variation such as that shown, for example, in the middle panel of in FIG. 1, is one method which indicates the presence of an indel region associated with a polymorphic region between related nucleic acid sequences. A local concentration of sequence variation can range from two to many polymorphic sites concentrated within a particular location of one of the suspected heterozygous alleles. The actual number of polymorphic sites will depend on the flanking region sequence and the method chosen by the one skilled in the art. For example, automated computer process such as Phred, Phrap and PolyPhred will be more sensitive than manual visualization. A local concentration of polymorphic sites can be identified by searching the database for these sequence characteristic parameters. Alternatively, programs well known in the art can generate annotated tags to graphically indicate a polymorphism. PolyPhred is one example of a program that can generate such annotated tags. Increased frequency of polymorphic tags in a localized region of a displayed sequence indicates a local concentration of polymorphic sites.

Additionally, a user can modulate settings to increase detection of the number of likely polymorphic sites. Increasing assignments of polymorphic sites by, for example, PolyPhred can result in a concomitant decrease in the confidence level of the actual nucleotide sequence. Possible errors incurred due to decrease confidence levels can be cured in subsequent steps of the method of the invention. For example, indel regions can be identified by multiple alternative criteria and either employed separately or together with the above method for determining the occurrence of criteria indicating the presence of an indel region. Therefore, one object for lowering threshold detection of possible polymorphisms is to ensure that all or most regions which can be suspected of containing an insertion or deletion are identified.

Another criteria indicating the presence of an indel region can consist of determining the occurrence of criteria indicating the presence of an indel region. Therefore, one object for lowering threshold detection of possible polymorphisms is to ensure that all or most regions which can be suspected of containing an insertion or deletion are identified.

Another criteria indicating the presence of an indel region can consist of determining the occurrence of proximal regions of unaligned sequence obtained from mated complementary sequence reads. An unaligned sequence obtained from mated complementary sequences reads refers to those regions of an alignment where the sequence is non-identical for at least 2 or more nucleotides. The greater the number of bases that are unaligned, the more likely that the sequences is an insertion or deletion rather that a SNP or multiple nucleotide substitutions.

As with identification of a local concentration of polymorphic sites, proximal regions of unaligned sequence obtained from mated complementary sequences can similarly be identified by, for example, automated or manual searching for polymorphic sequence regions containing these characteristics or by graphical output of annotated sequence tags. For example, proximal misalignments of mated reads can result in lower quality of the sequence data. Automated programs such as Phred, Phrap and Polyphred can characterize such sequences as low quality regions and label them as data needed or with a graphical data need tag.

An additional criteria indicating the presence of an indel region can consist of single sequence reads having unaligned sequence distally located to unaligned sequence positions of two or more related nucleic acids. As with proximally located unaligned sequence from complementary reads, distal regions of single sequence reads that become unaligned also are indicative of an indel sequence. Similarly, automated programs such as Phred, Phrap and PolyPhred can mark such regions with a characterizing parameter in the database. Alternatively, a graphical display output can be utilized which provide annotated tags indicating that the sequence within the distal region requires further data.

Once identified, related nucleic acids suspected of containing an indel region can be marked or tagged and then further analyzed in the methods of the invention to determine the actual sequence of the inserted or deleted sequence. Moreover, the methods for determining indel region sequences also are applicable for determining the actual nucleotide sequence of a substitution between two or more related nucleic acids.

The invention also provides a method of determining the sequence of an allele containing an indel region within a set of related nucleic acid sequences. The method consists of: (a) identifying a nucleic acid containing an indel region within two or more related nucleic acid sequences; (b) generating a consensus sequence within said indel region for said two or more related nucleic acid sequences; (c) identifying a matching string to said consensus sequence within at least one of said two or more related nucleic acid sequences, and (d) subtracting said consensus sequence from said two or more related nucleic acid sequences, the presence or absence of a unique sequence in one of said related nucleic acid sequences indicating the presence of an actual indel region.

As with the above described automated method for identifying different polymorphisms and indel regions, the methods of determining the sequence of an indel region within related nucleic acid sequences similarly can be employed independently or used in combination with other sequence discovery, analysis and data management system, such as is described further below. The methods for determining the sequence of an indel region is a component of the submodule “Indel Finder” within Module 4 of FIG. 4. Similarly, the methods for determining the sequence of an allele containing an indel region can be used alone or in combination with the previously described methods of the invention for identifying a plurality of different polymorphisms.

Sequences identified as containing an indel region by two or more of the above criteria, or other criteria known in the art, also can be further analyzed to determine the actual nucleotide sequence of the inserted or deleted sequence. Determination of the indel sequence yields the actual genotype of the related sequences that are compared and therefore, results in the differentiation of various alleles obtained from a sample population. Thus, the methods of the invention can be advantageous applied for determination of a plurality of heterozygous alleles within samples in an high-throughput automated system.

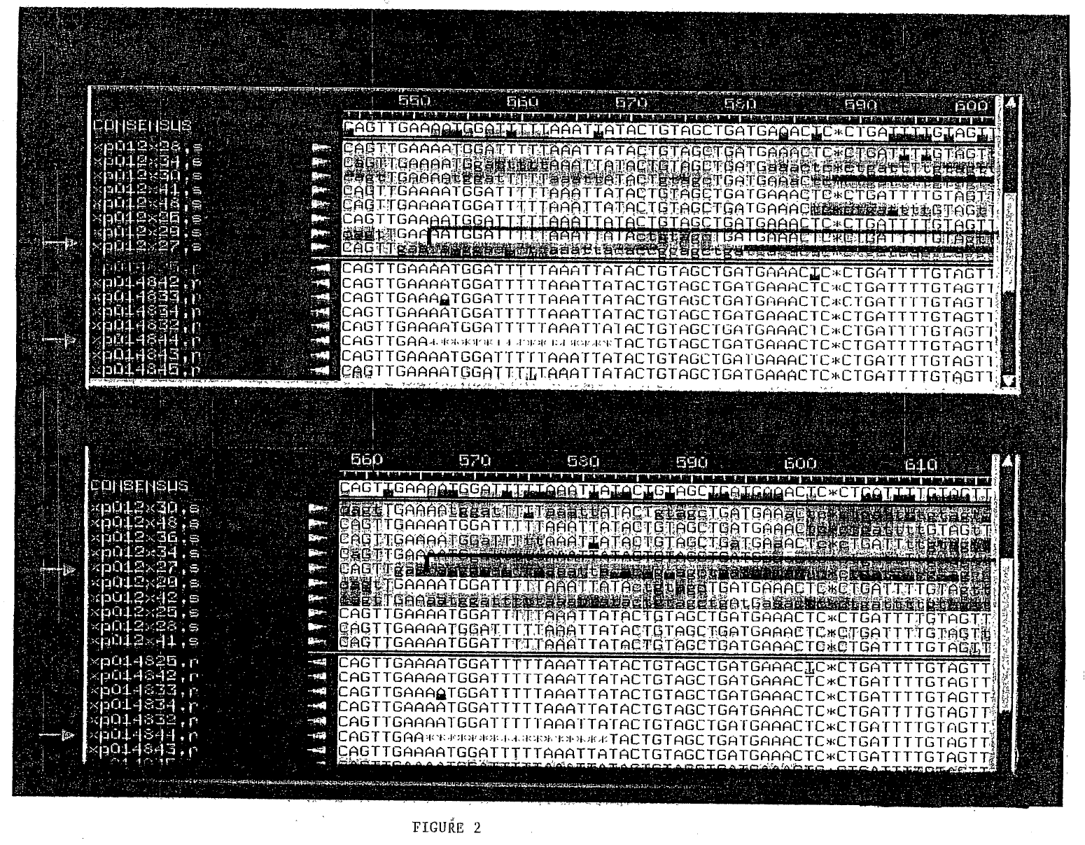

For both identifying indel regions and obtaining a consensus sequence, this method of the invention can advantageously utilize Polyphred program to automatically call genotypes for all sequences. Additionally, this method can function independently from the need for neighboring homozygous indel read sequences. Briefly, due to frameshifting caused by indel regions, sequence quality in such locations can be too low to be called by the Phrap/Polyphred program or other automated programs known in the art. Similarly, low quality regions within a frameshifted region can also be difficult to manually evaluate. For example, shown in FIG. 2A is one example of such a region where the sequence quality is sufficiently low that automated programs will not identify and place polymorphism tags in the frameshifted region. As exemplified below, the golden tag region and grey letter region in read xp012x27.s of FIG. 2A corresponds to an indel region but is not identified as polymorphic by the default settings of Polyphred. A color drawing of FIG. 2 is attached as Exhibit B.

The method for determining an indel region sequence can identify possible indel regions by, for example, lowering the threshold of the Phrap and Polyphred system to force these programs to call genotypes for all sequences. Similarly, the threshold for sequence quality for other automated systems also can be lowered to force genotype calls for all sequences within the analysis. For example, shown in FIG. 2B is the same sequence region of FIG. 2A where the threshold criteria for identifying genotypes has been lowered. Under these conditions, the indel region is identified by the the removal of the golden tag region and grey letter region and the appearance of new polymorphism tags in read xp012x27.s.

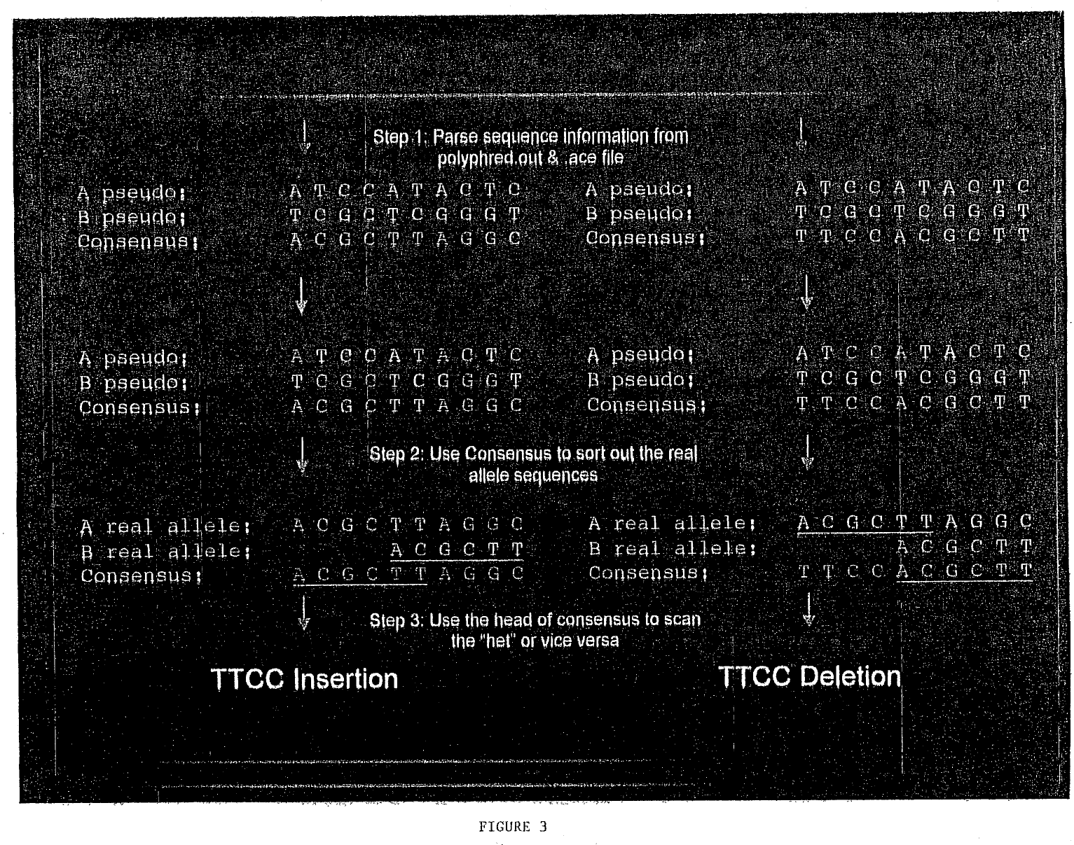

Once an indel region is identified between two or more related nucleic acid sequences, a consensus sequence within the indel region can be generated. Such a consensus sequence can be obtained, for example, directly from the available features of PolyPhred or from other programs known in the art or described above. Following obtaining a consensus sequence, both consensus sequence information and a “pseudo-genotype” information for the indel region can be extracted from the sequence data as shown in FIG. 3, step 1. A pseudo-genotype corresponds to a genotype obtained by, for example, lowering the threshold criteria for sequence quality in one or more read sequences. A color drawing identical to FIG. 3 is attached as Exhibit C.

For example, where insertions and deletions exist within related sequences, a direct sequence reading from an automated program is mixed up with sequences from two alleles. The actual sequences for the two alleles can be sorted out using, for example, the consensus sequence information as shown in FIG. 3, step 2. Briefly, read sequences corresponding to the pseudo-genotype sequences can be scanned with the indel region consensus sequence to identify a matching string. Any of a variety of sequence homology algorithms can be used for such scans including, for example, a simple string search or more complex heuristic algorithms. Once a matching string is identified, the consensus sequence can be subtracted from the combined sequences of the related pseudo-genotypes to indicate the presence or absence of an actual indel sequence. FIG. 3, step 2 and Exhibit C maps out identification of the actual allele sequences by separating sequences corresponding to the consensus sequence into different alleles.

For the example shown in FIG. 3, after subtracting the consensus sequence from a combined sequence corresponding to both pseudo-genotypes, the actual genotype of the heterozygous allele can be identified. In the specific example of the insertion sequence in FIG. 3, allele B is identified and for the deletion in FIG. 3, allele A is identified. To identify the alternative heterozygous allele, the beginning portion of the consensus sequence, shown as the underlined sequence ACGCTT in FIG. 3, can again be used to scan that allele. As shown in FIG. 3, for the insertion case, a matching string ACGCTT will be found in the allele B after scanning and a TTCC insertion can be identified. Where, for example, the first round of scanning does not find a match, the beginning portion of the alternate allele can be used to scan the allele that is identical to the consensus. Similarly, if a match is found, a deletion can be identified.

Automation of the above described methods for identifying a nucleic acid containing an indel region and for methods of determining the allele sequences of an indel region can be implemented following, for example, the logic in the pseudo code set forth below. Both pesudo codes are described in java with the pseudo code for the former method termed “FindIndels.java” and for the latter termed “FindIndelSequence.java.”

FindIndels.java:

Open ace file
Get contigs
For each contig:

Get reads
For each read:
Get the ace tags
Get the phd file
Parse the phd file
Get the phd “Data needed” and “Polymorphism”
tags
Save read info

If searching for mate pairs:
For each read:

If read is complemented:

Get the read's mate
If distance between “Data needed” tags for the
reads < requested distance:

Add bases between “Data needed” tags to
the indels list

If searching for outliners:

Compute average start positions for “Data needed”
tags on forward and reverse ends of the contig
For each read:

If start position for this read's “Data
needed” is > average + requested distance:

Add “Data needed” tag start position ± a
few bases to the indels list

If searching for polymorphism concentration:

For each read:

Using a sliding window of size N, search for
regions of “Polymorphism” tag concentrations >
C
Add all bases of such regions to an array

Combine regions from all reads
Add all flagged bases to the indels list

Create a new ace file with indels tags added from the
indels list

FinIndelSequence.java:

Input consensus, read1, read2, and suspected indel start
position
If consensus[start]=‘*’:

While (not found and end < consensus lenghth)
If consensus[end]≠‘*’:

found=true

Else

end++

For each base from start to end:

If read1[base]=the consensus [base]:

Indel [base]=read2 [base]

Else

Indel [base]=read1 [base]

Return inserted sequence Indel

Else

Create a window of bases to match:
For each base from start to start + window
size:

If read1[base]=the consensus [base]:

Window [base]=read2 [base]

Else

Window [base]=read1 [base]

While (not found and end<start+scan length)

If Window consensus[end:end + window size]:

found = true

Else

end++

If found = true:

For each base from start to end:

If read1[base]=the consensus [base]:

Indel [base]=read2 [base]

Else

Indel [base]=read1 [base]

Return deleted sequence Indel

The above described methods for identifying a plurality of different polymorphisms, for identifying nucleic acids with indel regions and for determining the sequence of an indel region can be used alone or in combination with other sequence data management, mining or analysis systems. For example, these methods for identifying and determining the sequence of various types of polymorphisms can be incorporated separately or combined into one component of a larger sequence discovery and data management system having an overall function of obtaining and accurately determining large volumes of nucleic acid sequence information. The sequence information of such a larger system can be generated de novo or obtained from independent sources. Obtained sequence information can be analysed and processed and placed into a database together with information generated during the acquisition or analysis phase of the procedure. Such indexed sequence data and associated information can be accessed and manipulated or analyzed further in any of a variety of different ways to obtain useful outputs of accurate sequence data. An example of one such larger system having the overall function of directing the data flow from acquisition to accurately identifying and outputting nucleic acid sequences is shown in FIG. 4.

In one embodiment, the invention is directed to a Polymorphism or SNP Discovery System which functions to enhance the throughput and accuracy of polymorphism and SNP discovery laboratory processes. The system automates previously manual operations, including SNP identification and recording. For example, a Polymorphism Loader can be used to automate these process to substantially increase the number of polymorphic sequences which can be identified and analyzed per unit time compared to previous methods. Therefore, polymorphism identification and analysis can be routinely performed in high throughput formats on a daily or hourly basis. Programs employed in the Discovery System take advantage of publically available software well known in the art, including for example, PolyPhred 3.5 and Consed 10.0. The SNP Discovery System can be composed of an Oracle database and a series of external Java™ and Per1 modules responsible for parsing and importing external data, and directing the action of the PolyPhred assembly system.

Briefly, the SNP Discovery System can include a number of Modules which are responsible for augmenting the function of a broad range of SNP discovery processes. The process can be initiated, for example, via a Sample Submission Module, which is shown as Module 1 in FIG. 4. A Sample Submission Module can transmit data for core Statistics Loading and post processing steps while simultaneously loading data into a SNP Database (SNP DB).

The Statistics Loading and post processing steps component of the system are shown as Module 2 in FIG. 4. The Assembly Module of the system is shown as Module 3 and functions to extract data from the SNP Database and constructs sequence assemblies from the extracted data. The Assembly Module also makes the resultant assemblies available to the SNP Prospector Module. The SNP Prospector is shown as Module 4 and is a set of computational tools supporting SNP mining activities. Users can, for example, review, select and confirm automated SNP-calls as well as conduct more refined analysis using the subcomponent indelFinder, which identifies nucleotide sequence insertions and deletions.

Manipulation of data for the SNP Prospector Module can be performed by a polymorphism Loader. In addition to data obtained through initial sample submission by, for example, de novo analysis, SNP data also can be obtained from external sources including, for example, publically available SNP or polymorphism databases. Such external SNP sequence data can be entered into the SNP Database using a function supplied by the External SNP Module shown as Module 5 in FIG. 4.

Other functions contained within the SNP Discovery System of the invention include, for example, the three additional modules termed SNP Export Module, SNP LIMS, and SNP View. The SNP Export Module transfers data from the SNP Database into custom research databases for project specific efforts. The SNP Export Module can enable the use of the SNP Discovery System by internal users or can be modified to accommodate its use by external projects and contracts, for example. The SNP LIMS System component functions to process and manage all aspects of laboratory information management coordinating high-throughput data generation aspects of SNP discovery. SNP View is a component that allows the visualization of the SNP of interest within its gene context.

The SNP Discovery system functions to manage data downstream of DNA sequencing pipeline and processing. The Discovery System can extract, store, and process pipeline sequencing results and can suggest actions based on SNP analysis of the sequencing results. Specific examples of suggested actions include, for example, re-sequencing, reconfiguration or new template preparation. The system also contains a logic that creates and populates the data structure required for polyphred assembly. Moreover, the system can additionally control, for example, the assembler (phredphrap) and automatically parse and upload results into a database. Results can be presented to end users through a graphics user interface (GUI), for example. Results also can be used to conform, discard, or edit automated SNP genotype calls with concomitant storage and reporting of the results. Exemplary tasks of the SNP Discovery System include the retrieval of sequence statistical information from the sequencing system; reprocessing of statistics in a customized manner, including on a project-by-project basis; automatic assembly of reads based on criteria established by the sequencing project; facilitating the identification and documentation of SNPs, and reporting obtained information to users.

The SNP Discovery System can receive initial information on samples as early as submission for sequence processing. For example, the SNP Discovery System can be implemented immediately after the sequencing of samples, and can assembly the sequences and analyze the assemblies on a per assembly group basis. The SNP Prospector Module of the system can be implemented for tagging assemblies of contiguous DNA sequences for either SNPs or indels which can be entered into a database. The SNPs or indels also can be confirmed by other methods or procedures, including for example, conformation by human review. The Discovery System also has the capability to sort assemblies by the presence of tagged SNPs or indels.

The SNP Discovery System is described below with reference to exemplary embodiments. However, it is understood that those skilled in the art will know, or can determine, that the system architecture and configuration as well as the functions of the modules and components can be simulated or performed by other structures and logic well known to those skilled in the art. Therefore, using the teachings and guidance described herein, functional substitutions and minor modifications of the structures and components described below for the SNP Discovery System can be made by those skilled in the art and still be encompassed by the Discovery System of the invention. For example, the SNP Discovery System is described with reference to the identification of SNPs. However, those skilled in the art will understand that the system is applicable to all types of polymorphisms in general as well as to other fields of genomics. For example, the SNP Discovery System can be implemented in the field of comparative genomics because the relatedness of compared nucleic acids sequences is a central issue to this type of discovery science.

The general system architecture and configuration of the SNP Discovery System is such that it can function as both a polymorphism data management system as well as a tool for the efficient mining of polymorphisms such as SNPs and indels automatically or through a human operator via the SNP Prospector Module. Therefore, this system functions as a framework to collect, collate, manage, monitor and confirm polymorphism data. The SNP Discovery System is extensible and designed to integrate data from SNPLIMS and to further functions such as SNP annotation. The SNP Discovery System also can be easily modified to accommodate changes in polymorphism detection technology. Finally, the system can serve as a data source for functions such as SNP Annotation and discovery tool integration.

Briefly, the architecture of the SNP Discovery System is based on three-tier architecture. However, other architectures known to those skilled in the art can similarly be employed using the teachings and guidance provided herein. The first tier contains an Oracle RDBMS that can be used for data persistence. The Oracle RDBMS or other comparable system can house the SNP database (SNP DB) schema. This database can be accessed by a variety of means well known to those skilled in the art, including for example, through standard SQL (Structured Query Language) using JDBC (Java Database Connectivity), ODBC (Open Database Connectivity), or PERLDBI (Perl Database Interface). Other related databases such as SNP Discovery system (called iDRLIMS or iSNPLIMS) can also be contained on the same server, making it efficient to tie data between databases for access by applications and users.

A second tier consists of the mapping of the data rows from the database to objects and is managed by, for example, an application server. One useful server of this type can be, for example, a WebObjects application server for read/write access to the database by users. WebObjects is a flexible, scalable platform to develop and deploy client server applications using, for example, either web-based access or Java client application access or both. The technical specifications for WebObjects application servers are well known to those skilled in the art.

The third tier contains a client application in the form of Java applets, or their equivalents, that can retrieve data and present it to users. Such Java Client applets can run in a web browser or applet runner in any Java 1.18 or higher environment. Moreover, no application or server specific files are necessary apart from the JRE (Java Runtime Environment). Currently, the applications can be accessed using Internet Explorer on a Macintosh OS 9.0 (and lower) and using AppletViewer on NT and MacOSX. Additionally, swing classes provide platform-specific look and feel.

With reference to SNP platform-specific architecture for the SNP Discovery System, data flow and relationship to other databases can be achieved by a combination of automated scripts and GUIs. Data flow specifications are entirely database centric, for example, all the necessary data for scripts to retrieve data and operate helper programs are stored in the database. However, non-centric data flow specifications also can be employed using logic and algorithms well known to those skilled in the art. Automation of the SNP Discovery System of the invention can be implemented following, for example, the logic in the pseudo code and select statements set forth below in Table 1 for data distribution and assembly process flow of the system. An exemplary description of data flow also is set forth below following Table 1.

Data Distribution and Assembly Process Flow

| | |
|---|---|
| Pseudo code | Select statements |

Main

Is this an ‘auto’ assembly or a ‘manual’ assembly?
If ‘auto’ {

| | |
|---|---|
| Select all asms that are ready | select * from wfele1Values where wfele1State=‘procready’ |
| Error check if 0 rows returned | |
| Save wfele1Id, clientId, authuserId for each asm | |
| For each asm | |

| | |
|---|---|
| Select asm properites row | select * from wfele1Props where asm_id = wfele1Id and |

| | wfele1Env = ‘prodn’ and |
|---|---|
| | wfele1Status = ‘active’ and |
| Error check if 0 rows returned for asm | Term Timestamp is not expired |
| Error check i8f > 1 rows returned for asm | |
| Save from wfele1Props: wfele1pRename, | |

wfele1pRunparam, wfele1Runstate,
wfele1pId, refSeqId

}
If ‘manual’ {

| | |
|---|---|
| Select asm specified | select * from wfele1Values where wfele1Value =$asm_name ar |

| | wfele1State = ‘procready’ and |
|---|---|
| Error check if 0 rows returned | wfele1Status = ‘active’ |
| Save wfele1Id, clientId, authuserId for each asm | |

| | |
|---|---|
| Select asm properties row | select * from wfele1Props where $asm_id = wfele1Id and |

wfele1Env = ‘test’ and
wfele1Status = ‘active’ and
term Timestamp is not expired

Error check if 0 rows returned for asm
Error check if>1 rows returned for asm
Save from wfele1Props: wfele1pRename,

wfele1pRunparam, wfele1Runstate,
Wfele1pId, refSeqId

}

For each assembly group {
Retrieve reference sequence

| | |
|---|---|
| Select reference sequence row | select * from wfwle2Props where wfele2Id = $refSeqId |
| Error chcek if 0 rows returned | |
| Save wfele2pSeq, wfele2pBounds 1a, | |

| | |
|---|---|
| wfele2pBounds1b | Note: allow for flexibility with bounds |

Validate refernce sequence for bad characters,

null value

Report errors
Valifdate bounds if null; use start=1, end=length
if null

Retrieve next serial number for asm

| | |
|---|---|
| Select key row | select * from snpKeyvalues |
| Increment value | |
| Update key row | update snpKeyvalues |
| Save value for this asm | |

Create asm location

| | |
|---|---|
| Select dataSet row for this asm | select * from dataSets where clientId = $cId |
| Save setLevel1-6 | |
| Retrieve serial number for asm | |
| Construct directory filepath | |

Save filepath

Create asm directories
Retrieve reads for asm

| | |
|---|---|
| Select read rows | select * from initSamps where initSamps.wfele1Id=$asm_id ar |

| | InitSamps.sampprild = sampResults.samppriId |
|---|---|
| | and |
| Error check if 0 rows returned | SampResults.postFinalstatus! =“discard” |
| Report any ‘eval’ rows returned | |
| Save only reads with‘accept’ status | |

SampResults.sampresultFilepath,
InitSamp.SampAlias1, 2, 3

Count reads that qualify for assembly and save

Rename reads feature

Check if wfele1pRename indicates rename
For each read in assembly group

Retreive read filepathname
If rename {
Change file name to stampAlias1
Copy scf file to chromat_dir directory }

If not rename {

Copy scf file to chromat_dir directory }

Run phredPhrap

Retrieve command line options for asm;

save wfele1Runparams

Build command line for system call, using Run params
Invoke system call
Trap and evaluate errors, if any

Create assembly group reports row

Retrieve save authuserId, clientId, wfele1Id,

wfele1Assemname (serial #)
wfele1rLoctation, wfele1rnNumbreads (read count),
wfele1rState (‘complete’), wfele1rStatus (‘active’)
wfele1rTimestamp (current date/time), wfele1pId

Insert into wfele1Reports

}
Evaluate errors from run
Email run status to users
Select email address of authorized users
Format email

| | |
|---|---|
| Mail | elect * from authUsers wher $cid= clientId |

Briefly, when a work request is submitted, data management in the SNP Discovery System is initiated. The work request can be similar to one which can be submitted for non-standard sequencing, except for SNP processing specific information such as assembly groups, reference sequences, and other sequence characteristic parameters associated with polymorphic regions and sequences. Upon arrival of processed sequences, which includes for example, results of SNP detection obtained by sequencing, or upon notification from a sequence processing and distribution module, a result loader program can be used to load these results into SNP DB. The result loader can additionally include, for example, functions directed to formatting the results for upload to SNP DB. Therefore the SNP database and applications can be insulated from changes upstream in the data flow. Such changes can include, for example, the replacement of sequencing processing or changes to that system.

Post processing scripts can process sequence processing results to SNP Discovery System-specific calls, which can then be used, for example, by automated assembly scripts. Automated assemblies can be created based on parameters specified in the database. Following assembly, a report is written to the database for user access. Next, an automated SNP scoring program can parse each assembly and write the parsed SNPs into a table called SnpPrgIdents. This table can hold parsed SNPs from both internal and external sources.

The results can be reported by, for example, viewing and approval by a user of the automatically scored SNPs and then recordation in a table called snpuseridents. Final reports can be generated from this collated SNP data in conjunction with related data from SNP DB or other data sources. One point of interface for updates between SNP DB and an external database system can be the Results Loader. Using this point of contact minimizes changes that need to be made to the SNP DB.

The timing and coordination of database updates can occur and will depend on the need and availability of the various users and the projects on implemented by the system. For example, the SNP Discovery System can be programmed for automated processing to occur once in a 24 hour period under normal use conditions. Automated processing consists of a linear series of programs and scripts that depend on the completion of the preceding process. A means to invoke the automated programs and scripts manually can be implemented through modifications well known to those skilled in the art. Such manual implementation can be important in the event that automated processing fails, was interrupted or was unable to commence. For SNP sequence processing to occur, sequence processing should be completed before the start of the SNP Discovery System.

System components requirements for the SNP Discovery System include, for example, GUI components for an administrator and for users, automated processing components and reporting components. Other components well known to those skilled in the art for augmenting, combining with, or modifying the function or efficiency of the SNP Discovery System can additionally be included as system components for the automated process of the invention. Those skilled in the art will know, or can determine, how to incorporate such additional systems components or modify those set forth above and described below given the teachings and guidance provided herein.

GUI system components for an administrator of the Discovery System can include, for example, components directed to new user and client profile, data sets definitions, workflow elements definitions, menu items definitions, transaction log view, key values or Snpkeyvalues set up and delete data by transaction identification. For example, the new user and client profile system component can allow, for example, an administrator to have the ability to add and update client information. The client information can include, for example, general information, as well as the ability to specify the post processing criteria which can be used by a post processing script. Post processing includes specifying the minimum number of reads required before assembly, the minimum VHQ (Very High Quality) for a read to be considered acceptable for inclusion in an assembly, and necessary workflow elements. Additionally, the system can allow an administrator to have the ability to add and update user information and assign or modify access permissions. Administrators can also be allowed by this system component to have the ability to create authorization strings, which define access permissions.

The data sets definitions system component can include, for example, the property of allowing an administrator to specify distribution levels at a subproject level. A workflow elements definition component can allow an administrator to have the ability to add and update workflow elements for a project per client requirements. The system component for key values or Snpkeyvalues, set up can include, for example, the function of allowing an administrator to have the ability to access key values whereas the system component for deleting data by transact identification can allow an administrator to have the ability to delete a set of data via the transact identification name.

GUI system components for a user of the Discovery System can include, for example, components directed to reference sequence set up, assembly groups set up, assembly reports view, sample submission, program-identified SNP view, or SnpPrgIdents, an user-identified SNP record, or SnpUserIdents, results view, login panel, GUIs access panel and accept and discard reads. For example, a reference sequence set up component can include the function for allowing a user to add and update reference sequences.

Briefly, an exemplary system can include two means for input of reference sequences. One means can be, for example, a low volume, manual method of input where the sequence can be entered from a GUI. An alternative means can include a file loader based algorithm where a GUI will accept a path to a file that has been preformatted for reference sequence upload. For both means, validation can be performed, for example, by including invalid characters such as carriage returns, null values which are impermissible for sequence bounds, and reference sequence names which can be unique for a given internal or external source.

An assembly groups set up system component for a user can include, for example, the ability of defining an assembly group either at the time of submission or prior to submission using a GUI. Each assembly group set up can be identified by an assembly group name that is unique within the dataset and can further be associated with, for example, a reference sequence that has been preloaded into the SNP database SNP DB. Assembly groups submitted at the time of sample submission can be newly created provided a previous entry for the assembly group does not exist. Assembly Groups have properties that associate a reference sequence and assembly parameters for automated assembly. One property of this system component can be to allow a user to create temporary assemblies. Such temporary assemblies will generally be constructed for testing purposes which can include changing run parameters and the inclusion and exclusion of reads. Reports for these assemblies can be written to the assembly reports table for a record of system activity. Moreover, any temporary assembly runs do not have to be carried over to SNP scoring processing, unless otherwise specified by the user. An administrator or a user can activate the assembly for SNP scoring processing when temporary assemblies are carried over to the SNP scoring processing.

The assembly reports view component provides automated program reporting to the database about the processing status of assemblies. Assemblies can be assigned, for example, serial numbers for tracking in the file system or other relevant information or parameters. The status of each assembly can be viewed through a GUI for an assembly group reports. Invalidating a specific assembly can be eliminated from further processing, including for example, having it dropped from SNP scoring processing.

A sample submission system component for a user can allow a user to submit samples, for example, through a user interface. One useful type of input can be, for example, a tab-delimited file. A non-standard submission file for the sequence processing submodule SEQMILL can be used for submission to the SNP Discovery System and a job identification code can connect a submission between SeqMill and the SNP Discovery System, for example. Sample submission can also include the function of inserting the assembly group row or rows. Other features of this system component can be, for example, to allow the user to assign multiple alias names to a read. Five to ten alias names is generally sufficient to maintain system efficiency and user flexibility. Generally, the first alias name can be used as a default for the primary sequence name.

The GUI system components for a user directed to program-identified SNP view, or SnpPrgldents, and to user-identified SNP record, or SnpUserldents, include the function of allowing a user to accept, discard or change the polymorphisms generated by polyphred that have been loaded into the database by the automatic processing. This user input can then be recorded, for example, in a separate space without affecting SNPs called by polyphred. The GUT can be modeled on the a filemaker GUI. Additional functionalities such as drag and select also can be included.

An accept and discard reads component also can be employed for automatically determining whether a read qualifies for inclusion in an assembly group. The logic and algorithms for such determinations are well known to those skilled in the art. Reads marked “accept” can be included in an assembly. The rules for such selections are described further below with reference to the post processing script. An additional function of this component allows a user to override a read's status.

A system component for automated processing also is included in the SNP Discovery System of the invention. The components for automated processing can include, for example, a results loader for core processing, a post processing component, an automated assembler, a component directed to inserting polymorphisms generated PolyPhred and find indels.

The results loader or core processing component includes, for example, functions for retrieving core statistics data on a read-by-read basis from sequence processing components such as SeqMill or SPEED. The retrieval can be set for essentially any interval but generally retrieving core statistics data once a day can be sufficient. The process can use sample list identification codes to retrieve a read's core statistics as well as that read's corresponding runfolder name and runfolder identification from SeqMill. In addition, the process can function to store the file path where the SCF file for the read can be located. Moreover, this information can be deposited, for example, into the SNP database.

A post processing component functions in two phases. One phase involves determining whether given reads should be included in an assembly. Criteria for this determination can be assigned on a project-by-project basis. Steps in the process include, for example:

Re-evaluation of a given read's status by comparing its VHQ value to a project-specific VHQ threshold: if the read's VHQ value is less than the threshold value, the read is assigned a status of ‘failed-seq’ (rather than ‘passed-seq’).

Identification of the read with the highest VHQ of all reads with same name: that read will be included in the assembly, while the lower VHQ reads will not be included.

Determination of a given read's pairing status: if a read possesses a corresponding mate (in the opposite direction), the pairing status is set to true.

And determination of the status of both paired reads. For example: (1) if forward and reverse passed, status is “both_dir_passed;” (2) if forward and reverse failed, status is “both-dir_failed;” (3) if forward failed, status is “forward_failed,” and (4) if reverse failed, status is “reverse_failed.”

Based on the above values, a given read can receive a final status value of “accept” or “discard” to determine whether it is included in an assembly or omitted. By default, a read will be included if it has a status of “passed_seq,” it has the highest VHQ of all reads with that name, and it possesses a mated read with a status of “passed_seq.”

The second phase of post-processing involves determining whether appropriate conditions have been met for a waiting assembly group to be assembled. If an assembly group has met the assembly conditions, which generally include a minimum set of available reads, a status value will be set in the database for that assembly group to signify to downstream processes that that group should be assembled.

An automated assembler component included in the SNP Discovery System can contain, for example, functions for identifying assembly groups that are ready to be assembled, retrieving the associated reference sequence and format in, for example, a FASTA phd file, retrieve the reads associated to an assembly group and assemble with the reference sequence, and identify an assembly group as completed. The location of the assemblies for a project also can be stored in the database. The system also can insert, for example, assembly information into the database and omit assemblies that contain multiple contigs or reverse compliment reads.

The system component for insert polymorphisms generated by polyphred includes functions, for example, to load polymorphism data generated by PolyPhred once the assembly processes have been completed. The program can query the SNP database to identify recently completed assembly groups and their locations in the filesystem. For each completed assembly group, the program also can parse that assembly's ace and polyphred files and reprocess that information. These putative polymorphism calls for the assembly group can then be inserted into the database. Data which can be included, for example, consists of: contig name; sample id; unpadded contig coordinate; padded contig coordinate; polymorphism rank; genotype rank; unpadded read coordinate; padded read coordinate; 5′ sequence context; 3′ sequence context, and read alleles. Once the database has been updated, the assembly group status can be updated to reflect this new status. An exemplary description of the function and data processing for PolyPhred and the Polymorphism loader and their corresponding data relationships in the SNP database is set forth below.

Briefly, for a given region of interest within a gene, DNA from many different sources can be sequenced. Generally, 96 to 192 samples from different individuals comprise a panel of reads. After sequencing, the sequencing files are passed into phred for base-calling, phrap for assembly and polyphred for polymorphism determination. The assembly generated has a consensus sequence, a reference sequence and numerous read sequences stacked and aligned together. The consensus sequences is determined by Phrap to be the most likely sequence by taking the highest quality bases from the reads used to generate the assembly. The reference sequence is the known sequence reported for the gene and entered into the system by independent means. Generally, the reference sequence can be taken from a public database such as Genbank, for example, and entered into the system by a scientist performing the experiments. The read sequences are the sequences generated for each sample using, for example, the automated system of the invention. The polyphred executable file detects ambiguities in the read's peaks and reports these as “genotype calls” with an associated rank to indicate likelihood (e.g., G/A, C/T, etc). Polyphred also examines these ambiguities across all of the reads for a given position. It uses the genotype calls in aggegate to determine the consensus sequence call and its associated rank. The Polymorphism Loader loads this data, parses it together with the ace file and indexes it in the SNP database. The data relationships within the SNP database are set forth below in Table 2.

TABLE 2
PolymorphismLoader/SNPStor Relationships

| | Database | |
|---|---|---|
| Database Column Name | Description | Meaning |
| DATASETID | Foreign key | Data set primary |
| | from datasets | identifier (set by |
| | table | database) |
| DATASETPROPID | Foreign key | Data set properties |
| | from | primary identifier |
| | datasetprops | (set by database) |
| | table | |
| INITIMESTAMP | Timestamp | Date and time |
| | | loading began |
| INIAUTHUSERID | Foreign key | Authorized user |
| | from | primary identifier |
| | authUsers | (set by database) |
| | table | |
| INICLIENTID | Foreign key | Authorized client |
| | from | primary identifier |
| | authClients | (set by database) |
| | table | |
| PRGCALLBASECHANGE | Poly- | The polymorphism call |
| | morphism | on the consensus |
| | call | sequence (generated |
| | | by polyphred) |
| PRGCALLBASENUM | Padded | The position of the |
| | consensus | consensus |
| | position | polymorphism call |
| | | (calculated by |
| | | polymorphismLoader) |
| PRGCALLCONTIG | Contig Name | The name of the |
| | | contig being |
| | | processed (i.e. |
| | | Contig1, Contig2, |
| | | etc.) |
| PRGCALLINDELSEQ | This string | The insertion/ |
| | corresponds | deletion sequence |
| | to the indel | determined by indel |
| | sequence, | parser |
| | which | |
| | will be | |
| | determined | |
| | and loaded | |
| | by the indel | |
| | parser | |
| PRGCALLINDELVAL | The value | The position at which |
| | will be set | the indel occurs |
| | by the indel | |
| | parser | |
| PRGCALLRANKCONS | Poly- | The rand (1-6) |
| | morphism | assigned to the |
| | rank | polymorphism |
| | | assigned to the |
| | | consensus sequence |
| PRGCALLRANKREAD | Genotype | The rank assigned to |
| | rank | the genotype call for |
| | | the read (assigned |
| | | by polyphred) |
| PRGCALLREADCOORDPAD | Padded read | The padded position |
| | position | of the polymorphism |
| | | call in the read |
| | | (calculated by |
| | | polymorphismLoader) |
| PRGCALLREADCOORDUNPAD | Unpadded | The unpadded position |
| | read | of the polymorphism |
| | position | call in the read |
| | | (generated by |
| | | polyphred) |
| PRGCALLREADGENOTYPE | Genotype | The two base calls |
| | calls | within the read |
| | | (generated by |
| | | polyphred) |
| PRGCALLREADSEQ3P | 3′ sequence | The read sequence |
| | context | 3′ to the base call |
| | | (determined by |
| | | polymorphismLoader) |
| PRGCALLREADSEQ5P | 5′ sequence | The read sequence |
| | context | 5′ to the base call |
| | | (determined by |
| | | polymorphismLoader) |
| PRGCALLREFBASE | Reference | The base in the |
| | sequence | reference sequence |
| | base | which corresponds to |
| | | the consensus and |
| | | read calls |
| | | (determined by |
| | | polymorphismLoader) |
| PRGCALLSAMPSEQ | The entire | The text string |
| | read | representing |
| | sequence | the entire read |
| | | sequence (taken from |
| | | ace file via |
| | | polymorphismLoader) |

A further system component for automated processing also can be included in the system which invokes the functions of the SNP Polymorphism Loader. This system component also can invoke, for example, other related automated processing components such as the SNP Export component which functions to process and transfer data to other research or specialized databases. Implementation of one or all of these functions in the automated system can be performed through the use of wrappers or applications that processes, transforms or moves data within or between components of the system. A description of the functions and subcomponents of this system component is provided below.

Briefly, the flow of information can be from the assembled sequences into the SNP Polymorphism Loader for further analysis using, for example, the SNP database or to SNP Export, for example, for further distribution to research and specialized databases. As described previously, various data mining and analysis from these database can be implemented by the automated system of the invention. Alternatively, implementation of the automated system of the invention can be further modified or augmented through minor modification by methods well known tp those skilled in the art.

Once read sequences are assembled into assembly groups using, for example, the previously described data distribution and assembly script a wrapper can be used to invoke the SNP Polymorphism Loader. The wrapper can, for example, pass to the Polymorphism Loader the assembly group name and the destination directory for the output file. The wrapper performing this function within the automated system of the invention is termed SNPExport_wrapper.

An application, termed SNPExtract, is another component of the system and functions to parse the SNP Polymorphism Loader output file, retrieve data from SeqMill or other sequence processing components, and formats the data into a text file. The text file output can be subsequently imported into an Excel spreadsheet or other useful format for automated or manual manipulation. The SNPExtract application also can accept the assembly group name and the destination directory for the output file and can be invoked by the SNPExport_wrapper script described above. Once the SNPExtract output file is imported into Excel each user can, for example, enter and save their base sequence calls.

Further subcomponents can include, for example, an application which combines the above two sets of calls into another tab-delimited file or finalScore. This text file also can be imported into Excel, and the spreadsheet used for capturing and saving the final calls. Additionally, an application that creates a text file containing the calls and supplemental data items can be employed for evaluation and transfer of information into a specialized database other than the SNP database. This application is termed aspolySNPprep and also can function to run an allele frequency script. Finally, an application that parses the file containing the SNP call information, runs an allele frequency script, and loads the data into a database can be included in this further system component. An example of a database that can be implemented by this aspect of the automated system of the invention is the ASPOLY database, which is a database set forth in the description below which is interchangeable with the previously described SNP database. Other functions and relationships of the above components are described further below.

For example, the SNPExport-wrapper component can be employed to invoke the Polymorphism Loader and the SNPExport applications. The wrapper can be, for example, a script or other functional equivalent, scheduled under cron to run at preselected intervals after the data distribution and assembly script has completed. The wrapper scrip can check for assembly activity by looking for sequencing results in a relevant directory. If there has been any sequencing, the two applications will be invoked, one after the other. If there has not been any sequencing, execution can stop. Both applications can be passed the assembly group name and the destination directory for the output file and a destination directory, such as /snpexport, can be created by the wrapper script. Assembly should occur before the wrapper script can continue.

Automation of SNPExport_wrapper functions can be implemented following, for example, the logic in the pseudo code set forth below. As with any of the previously described logic or algorithms, various modifications well known to those skilled in the art can similarly be incorporated or substituted for the functions exemplified in the described codes and algorithms.

SNPEexport_wrapper Pseudo code:
Invoke by cron
Scan project incoming data_directory for dated directory matching
today's date

Include option to run for any date specified

If no sequencing for date run, exit
If sequencing,

Identify assembly groups touched by sequencing
Create directory/project/subproject/mutation/new_exon/
<asmgrpname>/<date>/snpexport/
For each assembly group

Invoke snpemonPolymorphismLoader passing
asmgrpname and destination directory
Invoke SNPExport

Log execution or lack of execution

As also described previously above, the SNPExtract component functions to parse the output text file from Polymorphism Loader, retrieve supplemental data items from SeqMill, and format the information into a text file or other equivalent that can be imported into a spreadsheet or other useful format. This application can accept the assembly group name and the destination directory for the output file as described above. Additionally, the output of SNPExtract can be, for example, sorted by the SNP's position in the consensus. Other outputs can additionally be generated using methods well known to those skilled in the art. The Polymorphism Loader output is formatted by read. The two read directions can be, for example, merged into a single line for the template in the SNPExport output file. Much of the data for the template can come from the data for the forward direction, with the polymorphism rank and genotype calls from the reverse direction ‘merged’ into the line, for example. Because reads of very high quality are assembled, some directions can be omitted in the assembly and the information can instead be provided from SeqMill or other sequence processing component. Additionally, the 3′ sequence context can be any uniquely identifying size and is generally about 20 bases long, where the first base is the SNP, followed by 19 bases. Finally, a user can receive an email of the output file or the location of the output file.

The output text file can contain, for example, the assembly group name and the following data items set forth in Table 3:

TABLE 3

| Column | Data | |
|---|---|---|
| 1 | Read name without | PolymorphismLoader text |
| | direction extension | file |
| 2 | Polymorphism call | PolymorphismLoader text |
| | | file |
| 3 | Unpadded reference | Calculate |
| | position | |
| 4 | Reference sequence | PolymorphismLoader text |
| | position | file |
| 5 | Reference sequence | PolymorphismLoader text |
| | base | file |
| 6 | Unpadded consensus | PolymorphismLoader text |
| | position | file |
| 7 | Padded consensus | PolymorphismLoader text |
| | position | file |
| 8 | Padded reference | PolymorphismLoader text |
| | sequence position | file |
| 9 | Polymorphism rank— | PolymorphismLoader text |
| | forward direction | file |
| 10 | Polymorphism rank— | PolymorphismLoader text |
| | reverse direction | file |
| 11 | Genotype rank | PolymorphismLoader text |
| | | file |
| 12 | Unpadded read position | PolymorphismLoader text |
| | | file |
| 13 | Padded read position | PolymorphismLoader text |
| | | file |
| 14 | 5′ sequence context | PolymorphismLoader text |
| | | file |
| 15, | Genotype calls—forward | PolymorphismLoader text |
| 16 | (2 columns) | file (possibly SeqMill |
| | | also) |
| 17, | Genotype calls—reverse | PolymorphismLoader text |
| 18 | (2 columns) | file (possibly SeqMill |
| | | also) |
| 19 | 3′ sequence context | PolymorphismLoader text |
| | | file |

Automation of SNPExtract functions can be implemented following, for example, the logic in the pseudo code set forth below. Similarly, various modifications well known to those skilled in the art can similarly be incorporated or substituted for the functions exemplified in the code described below.

SNPExtract Pseudo Code:
Run for assembly group passed on command line
Retrieve all reads for assembly group and store sorted, forward
direction before reverse direction no duplicates
Parse snpemonPolymorphismLoader output file

For each line

Store each data item into hash

Create tab-delimitated output file

For each read in the assembly group

Merge forward and reverse data together
Calculate unpadded reference position
Print the first 20 bases of the 3′ sequence
For any read not in the Loader output file, print
‘-’ for missing info.

Write output to /snpexport/

Email output file or location of file

In regard to the FinalScore component, one of its functions is to combine two sets of calls into another text file. This script can be invoked, for example, by the user and can be additionally implemented to not require any command line options. The output of the script can be imported into Excel, or other equivalent format, and the spreadsheet can be used for capturing the final calls. For example, during the scoring process, a user can remove a set of reads for a SNP. These reads are also removed form the spreadsheet because the user has determined that it is a false positive. When there are multiple users, it is frequently the case that one user removes a SNP and the other user does not. During the merging activity of this script, the two original calls can be maintained, but the calls of the other user are, for example, left blank.

Automation of FinalScore functions can be implemented following, for example, the logic in the pseudo code set forth below, including modifications thereof using the teachings and guidance provided-herein.

FinalScore.Pseudo Code:

Run finalScore script in /snpexport directory.

Parse both files containing calls and populate hash.

Sort hash.

Print to output file combining data for same templates or print ‘−’ for missing info.

For the AspolySNPPrep, which can be the last step in the evaluation and transferral of sequence and scoring information with the creation of a text file. The text file can be subsequently imported into a database such as ASPOLY by AspolySNPLoader. This script can gather the scoring information and insert rows into, for example, the following ASPOLY tables: assembly, assemblysnp, and seqgenotype. The relationship of the data item with respect to its source and destination location in the database is set forth in Table 4 below. This script also can be modified to access the SNP database as an alternative to text files.

TABLE 4

| Data Item | Source | Destination Location |
|---|---|---|
| Assembly group | Directory name | |
| name | | |
| Scorer | Blank | Assembly-scorer |
| Final scorer name | FinalScore output file | Assembly.scorerName |
| | after data entry | |
| Date scored | FinalScore output | Assembly.scoreDate |
| | filename extension | |
| Resequence | Default = no | Assembly.reSeq |
| (yes/no) | | |
| Assembly | Default = yes | Assembly.Polym |
| polymorphic | | |
| (yes/no) | | |
| Scored (yes/no) | Default = yes | Assembly.scored |
| Comments | Added later (noted | Assembly.comments |
| | here for completeness) | |
| Unpadded | SNPExtract output file | Assemblysnp.phrapbase |
| reference | | |
| position | | |
| Allele1 name | Allele frequency script | Assemblysnp.Allele1 |
| Allele2 name | Allele frequency script | Assemblysnp.Allele2 |
| Allele1 frequency | Allele frequency script | Assemblysnp.Freq1 |
| Allele2 frequency | Allele frequency script | Assemblysnp.Freq2 |
| Number of | Allele frequency script | Assemblysnp.NumPeople |
| chromosomes | | |
| in frequency | | |
| SNP name | Added later (noted | Assemblysnp.SnpName |
| | here for completeness) | |
| Amino acid | Added later (noted | Assemblysnp.AADelta |
| change | here for completeness) | |
| Comment | Added later (noted | Assemblysnp.Comment |
| | here for completeness) | |
| Well location | Sequenceplatefile.RowNum | Seqgenotype.rownum |
| Asthma internal | Sequenceplatefile.Ind | Seqgenotype.ind |
| id (template id) | | |
| Genotypes | FinalScore output file | Seqgenotype.genotype |
| | after data entry | |
| Confidence | FinalScore output file | Seqgenotype.confidence |
| (polyphred | after data entry | |
| ranking high/ | | |
| med/low) | | |

Additional functions include, for example, where the assembly has been determined not to be polymorphic, then the assembly table row can be added by the AsppolySnPPrep component but the Assemblysnp and Seqgenotype rows are omitted, for example. Finally, AspolySNPLoader can load the data prepared by aspolySNPPrep into ASPOLY.

Automation of AspolySNPPrep and AspolySNPLoader functions can be implemented following, for example, the logic in the pseudo codes set forth below, including modifications thereof using the teachings and guidance provided herein.

AspolySNPPrep Pseudo Code:

Parse input file to get following values

$AsmGrpname from file name
$PhrapBase
$ScorerName
$ScoreDate from file name
$Genotype

Set to null

$SNPName
$AADelta
$AsmSNPComment
$ReSeq
$Scored
$AsmComments
$Polym
$Confidence

Calculate

$WellLocation

Run allele script to get/set

$Allele1
$Allele2
$Freq1
$Freq2
$NumPeople

Retrieve lookup row

SELECT SeqPlateId, SeqAssayPrimerId, ExonId, GeneIdd,

RegionId,

RowNum, Ind, Alias3
FROM sequencplatefile
WHERE AssemblyGrp like “$AsmGrpName” AND Direction like
“forward”

(need to clarify where clause)
Set returned values to variables
Insert row into ASSEMBLY

INSERT into ASSEMBLY
(SeqPlateId, SeqAssay, PrimerId, GenId, RegionId, ExonId,
ScorerName, ScoreDate, ReSeq, Polym, Scored, Comments)
VALUES
($SeqPlateId, $SeqAssayprimerId, $GeneId, $RegionId,
$ExonId, $ScorerName, $ScoreDate, $ReSeq, $Polym,
$Scored, $Comments)

Retrieve AssemblyId

SELECT AssemblyId
FROM assembly
WHERE SeqPlateId = $SeqPlateId AND SeqAssayPrimerId =
$SeqAssayPrimerId AND GeneId = $GeneId AND RegionId =
$RegionId AND ExonId = $ExonId
Set AssemblyId = $Assemblyid

Insert into ASSEMBLYSNP

INSERT into ASSEMBLYSNP
(AssemblyId, SeqPlateId, SeqAssayPrimerId, GeneId,
RegionId, ExonId, PhrapBase, Allele1, Allele2, Freq1,
Freq2, NumPeople, SNPName, AADelta, Comment)
VALUES
($AssemblyId, $SeqPlateId, $SeqAssayPrimerId, $GeneId,
$RegionId, $ExonIdk, $Phrap?Base, $Allele1, $Allele2,
$Freq1, $Freq2, $NumPeople, $SNPName, $AADelta, $Comment)

Retrieve AssemblySNPId

SELECT AssemblySNPId
FROM assemblySNP
WHERE Assembly = $AssemblyId AND SeqPlLateId = $SeqPlateId
AND SeqAssayPrimerId = $SeqAssayPrimerId AND GeneId
=$GeneId AND RegionId = $RegionId AND ExonId = $ExonId

Set AssemblySNPId = $AssemblySNPId
Insert into SEQGENOTYPE

INSERT into ASSEMBLYGENOTYPE
(AssemblySNPId, AssemblyId, SeqPlateId, SeqAssayPrimerId,
GeneId, RegionId, ExonId, RowNum, Individual, Genotype,
Confidence)
VALUES
($AssemblySNPId, $AssemblyId, $SeqPlateId,
$SeqAssayPrimerId, $GeneId, $RegionId, $ExonId,
$WellLocation, $Ind, $Genotype, $Confidence)

The find indels system component includes functions for calculating possible indel positions in an assembly using tags from ace and phd files. For example, “data needed” tags signify regions at the ends of reads where data quality is low and “polymorphism” tags signify polymorphisms. Search criteria for indel regions can be, for example, selected from one or more of the following:

“Data Needed” Outliers: Search for “data needed” tags that start a significant distance li-om the average starting-point. A possible indel may lie at the starting point of the outlier. The user may specify the minimum distance that defines an outlier.

“Data Needed” Mates: Search for mated reads that have “data needed” tags with starting points close to one another. A possible indel may lie between the starting points. The user can specify the minimum and maximum distances between the starting points of the tags.

“Polymorphism” Concentration: Search for regions of high polymorphism tag concentration. The user can specify the window size and minimum concentration to search for.

When indels are found, a new ace file can be created, for example, to contain added contig tags describing the possible indel positions.

A system component for reporting also is included in the SNP Discovery System of the invention. The components for reporting can include, for example, processing logs, assembly summary report, pair success report, list of reads to check for polymorphisms, general statistical reporting and status reporting on discrepancies between databases. For example, a processing logs component can include a log of the status of some or all automatic processing activity. The logs can be written to a SNP DB table and accessible by, for example, an administrator or a user. In some cases, manual intervention can be required, followed by reassembly and loading into the database. The logs can include the following conditions and information.

Logging can include:

(1) Process completed, invoked by user, and date/time.

(2) Process failed, reason, invoked by user, and date/time.

(3) Error condition encountered for a subset of data processed, reason and date/time.

(4) Results for preprocessing, including projects processed, job ids updated, number of samples processed and date/time.

(5) Results for post processing, including projects processed, data set properties used in evaluation and date/time.

(6) Results for auto assemblies, including projects processed, plate names for reads, assembly group assembled, number of reads in assembly, location of assembly and date/time.

(7) Results for putative polymorphism calls, including projects processed, assembly groups evaluated and date/time.

Error reporting can include:

(1) Missing Reference Sequence for an assembly group.

(2) Multiple contigs in an assembly.

(3) Incomplete ace file, indicating failure during phrap run.

(4) Singlet condition, determined by phrap.

(5) Chemistry not in phredpar.dat.

(6) Read(s) missing result rows.

(7) Project setup incomplete.

(8) Inability to write to a directory location.

(9) Inability to write to the database.

(10) Attempt to insert duplicate rows.

(11) SCF file not found.

(12) Reversed reads in assembly.

Another component of the system is an assembly summary report which can allow a project to have the assembly summary report generated after activity has occurred for a batch of assembly groups. The assembly summary report can list, for example, the assembly groups in a batch and can indicate whether an assembly group does not require further sequencing, based on error rate and length, for example.

A further function of the reporting component can be a pair success report which can include, for example, a report of pair success statistics for a project. This statistical report cna be based on post processing pair status, for example. A additional component can be a report which lists reads to check for polymorphisms. General statistical reporting and status reporting on discrepancies between databases can further be included as functions of the reporting component of the system. For example, the SNP Discovery System, SeqMill, and SPEED all depend on some data between systems and if a process fails to “pull” or “push” data, inconsistencies can result. Therefore, read information can be periodically checked between databases and reported.

Certain software well know in the art can be used in the SNP Discovery System for easy of implementation and compatibility with a variety of automated sequencing procedures; For example the SNP Discovery System can employ the following software packages which are well know to those skilled in the art: Phred, Phred-qual, Phrap, PolyPhred, PhredPhrap and Consed. Alternatively, substitution of other programs which perform substantially the same function also can be employed in the SNP Discovery System of the invention. The role each of these programs play and their dependencies in the SNP Discovery System are set forth below.

Briefly, Phred and phred-qual can be used to generate core statistics. Phred also is employed in the assembly process to create the “phd” and “poly” files. PolyPhred functions to detect the polymorphism calls. PhredPhrap is a script provided in the consed package and can be employed, for example, to streamline the calling of scripts required for Consed. Such scripts can be further modified to provide flexibility for the SNP Discovery System. Consed provides the ability to manually view assemblies and can further be simulated and the output inserted into the database.

Throughout this application various publications have been referenced within parentheses. The disclosures of these publications in their entireties are hereby incorporated by reference in this application in order to more fully describe the state of the art to which this invention pertains.

It is understood that modifications which do not substantially affect the activity of the various embodiments of this invention are also included within the definition of the invention provided herein. And although the invention has been described with reference to the disclosed embodiments, those skilled in the art will readily appreciate that the various specific embodiments detailed are only illustrative of the invention. Therefore, it should be understood that various modifications can be made without departing from the spirit of the invention. Accordingly, the invention is limited only by the following claims.

What is claimed is:

1. An automated method of identifying a plurality of different polymorphisms within two or more related nucleic acid sequences, comprising:
(a) obtaining a data set comprising a nucleic acid sequence assembly and a plurality of sequence characteristic parameters associated with said assembly;
(b) indexing said nucleic acid assembly and said plurality of sequence characteristic parameters in a database;
(c) selecting a region of said nucleic acid assembly having sequence characteristic parameters indicative of a polymorphic sequence, and
(d) displaying two or more nucleic acid sequences of said region, said two or more sequences identifying different polymorphisms within said nucleic acid assembly.

2. The method of claim 1 wherein said data set further comprises a phd file and an ace file, or functional equivalent.

3. The method of claim 1 wherein said text file further comprises a nucleic acid sequence.

4. The method of claim 3 wherein said ace file further comprises sequence characteristic parameters selected from the group consisting of background ratio, peak height ratio, sequence quality, and rank.

5. The method of claims 4 further comprising selecting sequence characteristic parameters indicative of a single nucleotide polymorphism (SNP).

6. The method of claim 5, wherein said automated method further comprises an accuracy of SNP identification greater than about 90%.

7. The method of claim 1 further comprising a plurality of nucleic acid assemblies.

8. The method of claim 1 wherein said two or more related nucleic acid sequences further comprise alleles.

9. The method of claim 1 wherein said two or more related nucleic acid sequences further comprise heterozygous alleles.

10. The method of claim 7, further comprising displaying two or more nucleic acid sequences of said region indicative of a polymorphic sequence for a plurality of different nucleic acid assemblies.

11. The method of claim 1 further comprising identifying an indel region, said indel identification comprising the steps:
(a) identifying a nucleic acid within two or more related nucleic acid sequences suspected of containing an indel region, said nucleic acid containing one or more regions having a plurality of polymorphisms, and
(b) determining the occurrence of two or more criteria indicating the presence of an indel region associated with said one or more regions having a plurality of polymorphisms, said occurrence characterizing said nucleic acid as containing an indel region.

12. The method of claim 11, wherein said indel region further comprises an uncharacterized nucleotide sequence length.

13. The method of claim 11, wherein said criteria indicating the presence of an indel region further comprises determining a local concentration of a plurality of polymorphic sites within at least one of said related nucleic acid sequences.

14. The method of claim 11, wherein said criteria indicating the presence of an indel region further comprises determining proximal regions of unaligned sequence obtained from mated complementary sequence reads.

15. The method of claim 11, wherein said criteria indicating the presence of an indel region further comprises determining single sequence reads having unaligned sequence distal to unaligned sequence locations of two or more related nucleic acids.

16. The method of claim 1 further comprising determining the sequence of an allele containing an indel region, said sequence determination comprising the steps:
(a) identifying a nucleic acid containing an indel region within two or more related nucleic acid sequences;
(b) generating a consensus sequence within said indel region for said two or more related nucleic acid sequences;
(c) identifying a matching string to said consensus sequence within at least one of said two or more related nucleic acid sequences, and
(d) subtracting said consensus sequence from said two or more related nucleic acid sequences, the presence or absence of a unique sequence in one of said related nucleic acid sequences indicating the presence of an actual indel region.

17. The method of claim 16, wherein said unique sequence further comprises an actual indel region sequence.

18. The method of claim 16, further comprising a consensus sequence obtained from three or more related nucleic acid sequences.

19. The method of claim 16, further comprising a consensus sequence obtain from ten or more related nucleic acid sequences.

20. The method of claim 16, further comprising a consensus sequence obtain from twenty or more related nucleic acid sequences.

21. The method of claim 16, wherein the presence of said unique sequence further comprises an insertion sequence.

22. The method of claim 16, wherein the absence of said unique sequence further comprises a deletion sequence.

23. The method of claim 16, wherein said steps further comprise an automated process.

24. The method of claim 16, further comprising identifying said matching string by a string search or heuristic algorithm.s

25. The method of claim 1 further comprising displaying said sequence characteristic parameters as annotate tags.

26. A method of identifying a nucleic acid containing an indel region within a set of related nucleic acid sequences, comprising:
(a) identifying a nucleic acid within two or more related nucleic acid sequences suspected of containing an indel region, said nucleic acid containing one or more regions having a plurality of polymorphisms, and
(b) determining the occurrence of two or more criteria indicating the presence of an indel region associated with said one or more regions having a plurality of polymorphisms, said occurrence characterizing said nucleic acid as containing an indel region.

27. The method of claim 26, wherein said associated region having a plurality of polymorphisms further comprises an indel region.

28. The method of claim 26, wherein said related nucleic acid sequences further comprise alleles.

29. The method of claim 26, wherein said related nucleic acid sequences further comprise heterozygous alleles.

30. The method of claim 26, wherein said indel region further comprises an uncharacterized nucleotide sequence length.

31. The method of claim 26, wherein said criteria indicating the presence of an indel region further comprises determining a local concentration of a plurality of polymorphic sites within at least one of said related nucleic acid sequences.

32. The method of claim 26, wherein said criteria indicating the presence of an indel region further comprises determining proximal regions of unaligned sequence obtained from mated complementary sequence reads.

33. The method of claim 26, wherein said criteria indicating the presence of an indel region further comprises determining single sequence reads having unaligned sequence distal to unaligned sequence locations of two or more related nucleic acids.

34. The method of claim 26, wherein said steps further comprise an automated process.

35. A method of determining the sequence of an allele containing an indel region within a set of related nucleic acid sequences, comprising:
(a) identifying a nucleic acid containing an indel region within two or more related nucleic acid sequences;
(b) generating a consensus sequence within said indel region for said two or more related nucleic acid sequences;
(c) identifying a matching string to said consensus sequence within at least one of said two or more related nucleic acid sequences, and
(d) subtracting said consensus sequence from said two or more related nucleic acid sequences, the presence or absence of a unique sequence in one of said related nucleic acid sequences indicating the presence of an actual indel region.

36. The method of claim 35, wherein said unique sequence further comprises an actual indel region sequence.

37. The method of claim 35, wherein said related nucleic acid sequences further comprise alleles.

38. The method of claim 35, wherein said related nucleic acid sequences further comprise heterozygous alleles.

39. The method of claim 35, wherein said indel region further comprises an uncharacterized nucleotide sequence length.

40. The method of claim 35, wherein said identification of said indel region further comprises determining a local concentration of a plurality of polymorphic sites within at least one of said related nucleic acid sequences.

41. The method of claim 35, wherein said identification of said indel region further comprises determining proximal regions of unaligned sequence obtained from mated complementary sequence reads.

42. The method of claim 35, wherein said identification of said indel region further comprises determining single sequence reads having unaligned sequence distal to unaligned sequence locations of two or more related nucleic acids.

43. The method of claim 35, further comprising a consensus sequence obtained from three or more related nucleic acid sequences.

44. The method of claim 35, further comprising a consensus sequence obtain from ten or more related nucleic acid sequences.

45. The method of claim 35, further comprising a consensus sequence obtain from twenty or more related nucleic acid sequences.

46. The method of claim 35, wherein the presence of said unique sequence further comprises an insertion sequence.

47. The method of claim 35, wherein the absence of said unique sequence further comprises a deletion sequence.

48. The method of claim 35, wherein said steps further comprise an automated process.

49. The method of claim 48, further comprising identifying said matching string by a string search or heuristic algorithm.

50. An automated system for identifying a plurality of different polymorphisms within two or more related nucleic acid sequences, comprising:
(a) a sample submission module capable of transmitting data;
(b) a core statistics loading and post processing module containing sequence characteristic parameters;
(c) an assembly module capable constructing sequence assemblies from sequence database extracted data;
(d) a SNP prospector module capable of identifying polymorphisms;
(e) a polymorphism loader submodule capable of parsing polymorphic region sequence and sequence characteristic parameters from sequence assemblies;
(f) a SNP database structured to contain the information produced in steps (a) through (e), and
(g) an output module for display or further manipulation of specified data in step (f).

51. The system of claim 50, further comprising data transmission to a Core Statistics Loading and Post Processing Module or to a SNP database.

52. The system of claim 50, wherein said database in step (c) further comprises a SNP database.

53. The system of claim 50, wherein said polymorphisms in step (d) further comprise a SNP or an indel.

54. The system of claim 50, further comprising an External SNP module capable of importing nucleic acid polymorphism sequence information from external sources.

55. The system of claim 54, wherein said external source further comprises a public database.
